(19) United States
(12) Patent Application Publication
(10) Pub. No.: US 2023/0232883 A1
(43) Pub. Date: Jul. 27, 2023

(54) METHOD FOR IMPROVING THE TECHNOLOGICAL AND NUTRITIONAL PROPERTIES OF GLUTEN-FREE FOOD MATRICES

(71) Applicant: FAVERO ANTONIO SRL, PADOVA (IT)

(72) Inventors: Carlo Giuseppe RIZZELLO, Bari (IT); Alessia GIANNONI, Costa Bissara (VI) (IT); Ilaria DE PASQUALE, Mesagne (BR) (IT); Loredana FAVERO, Villatora di Saonara (PD) (IT)

(73) Assignee: FAVERO ANTONIO S.R.L., PADOVA (IT)

(21) Appl. No.: 18/012,127

(22) PCT Filed: Jun. 22, 2021
(86) PCT No.: PCT/IB2021/055487

§ 371 Date: Dec. 21, 2022

(30) Foreign Application Priority Data
Jun. 23, 2020 (IT) 102020000014995

(51) Int. Cl. A23L 33/135; C12N 1/20; A23P 10/40; A23L 7/104; A23L 11/50

(52) U.S. Cl. CPC A23L 33/135; C12N 1/205; A23P 10/40; A23L 7/104; A23L 11/50; C12R 2001/24

(57) ABSTRACT

A method is provided for improving the technological and nutritional properties of gluten-free food matrices based on a fermentation protocol using selected lactic bacteria as starters. The bacteria used are Lactobacillus plantarum strain DSM 33412 and Lactobacillus brevis strain DSM 33413. The method is useful in the processing of gluten-free flowing food material.

### FIELD OF THE INVENTION

The present invention relates to a method for improving the technological and nutritional properties of gluten-free food matrices based on a fermentation protocol using selected lactic bacteria as starters.

The method of the invention finds application in the processing of gluten-free food material.

### BACKGROUND

Flours are an important and relevant component of everyone's diet and are used to create a large number of food products.

Flours are generally produced from cereals, some of which however contain gluten.

Gluten is a protein complex originating from prolamines and glutelins, two groups of proteins found in cereals, including wheat, barley, rye and spelt. Gluten gives doughs important characteristics such as viscosity, elasticity and cohesion during and after baking. Therefore, the quantity and quality of gluten in a flour is an important indicator of its suitability for baking.

However, gluten is one of the least digestible proteins for the human intestine and individuals with coeliac disease are unable to digest it. The only known treatment to date for individuals with coeliac disease is a gluten-free diet, which must be very strict, since the introduction of even small amounts of this substance into the body is enough to trigger the symptomatic response typical of the disease, mainly related to a progressive atrophy of the intestinal villi with consequent problems related to malabsorption.

For example, according to the standards of the Italian Coeliac Association, products with a gluten content of less than 20 ppm can be considered suitable for a coeliac; in other countries, products with a gluten content of up to 100 ppm can be considered suitable.

To meet these needs, the use of gluten-free cereal-based flour or food products for coeliacs is becoming more widespread.

However, gluten-free flours are difficult to work with and commercial gluten-free products suffer from these effects.

In order to partly overcome these drawbacks and to improve the workability of the doughs, an attempt has been made to use admixtures of gluten-free cereal flours, pseudocereals and/or legumes.

From a nutritional point of view, gluten-free flours, such as those obtained from legumes, are an excellent source of high biological value protein, carbohydrates and dietary fibre. In addition, they provide many essential amino acids, vitamins, minerals, oligosaccharides and phenolic compounds (Campos-Vega et al., 2010. Food Research International, 43, 461-482; Roy et al., 2010. Food Research International 43, 432-442). Frequent consumption of legumes is considered an effective way to decrease the risk of cardiovascular disease (CVD) (Flight and Clifton, 2006. European Journal of Clinical Nutrition, 60, 1145-1159), type 2 diabetes mellitus (Jenkins et al., 2012. Archives of Internal Medicine, 172, 1653-1660), some types of cancer (Feregrino-Perez et al., 2008. European Journal of Clinical Nutrition, 60, 1145-1159), overweight and obesity (Mollard et al., 2012. British Journal of Nutrition, 108, 111-122).

Although there are these potential beneficial effects, worldwide consumption of legumes is declining (Kohajdová et al., 2013. Chemical Papers, 67, 398-407) and is below the recommended dose (McCrory et al., 2010. Advances in Nutrition, 1, 17-30).

Among the reasons for their limited large-scale use is the fact that legumes, in particular, have high concentrations of certain anti-nutritional compounds including:
raffinose (and other alpha-galactosides such as verbascose and stachyose), oligosaccharides that are not digestible by humans and can reach the intestinal tract where they are fermented by the intestinal microbiota, resulting in gas and flatulence;
trypsin inhibitors, responsible for limited protein digestibility;
phytic acid, a compound that can complex minerals and proteins, reducing their bioavailability;
saponins and condensed tannins;
vicin and convicin (found in fava beans), compounds that can cause favism in genetically predisposed individuals (Rizzello et al., 2016. Scientific Reports, 6, 32452).

There is therefore a need to provide gluten-free flowing flowing material that does not have the drawbacks mentioned above.

According to the most recent scientific literature, a valid option for improving the sensory and functional quality of oven-baked leavened products fortified with legumes is the use of fermentation.

Fermentation processes of flours from unconventional matrices, such as pseudocereals and legumes, can be effectively conducted with the use of lactic bacteria (Gobbetti et al., 2019. Critical Reviews in Food Science and Nutrition, 1,1-16). These microorganisms, whose use in food biotechnology goes back a long way, are considered QPS by EFSA (Qualified Presumption of Safety) and can be used without restriction in food production processes.

In addition, natural fermentation can reduce the glycaemic response, increase protein digestibility and improve the bioavailability of compounds (Gobbetti et al., 2019. Critical Reviews in Food Science and Nutrition, 1, 1-16).

However, a selection of biotypes with the appropriate metabolic characteristics is indispensable for obtaining specific and appreciable results, such as the partial or complete degradation of anti-nutritional compounds.

### Definitions

Within the scope of the present invention, “granular material” means food material in the form of whole or broken grains, whether whole or not, or even non-powdery flours with an appropriate granulometry value preferably greater than 1000-1500 micrometers.

In the context of the present invention, “flowing flowing material” means material in the form of a non-powdery flour with a granulometry of between about 1000 and 1500 micrometers. Material in the form of flakes is also included in this definition.

In the context of the present invention, “starter” means one or more microorganisms used in a live and viable status for inoculating food biomass for transformation by fermentation into ingredients or foodstuffs or drink for food use. The term “starter” also refers to the preparation in liquid or solid form, fresh or frozen or lyophilised, containing a high cell density of the aforesaid micro-organisms in a live and viable form.

### DESCRIPTION OF THE DRAWINGS

FIG. 1 is a flow chart showing the various steps of the fermentation method.

FIG. 2. pH (A), lactic acid (mmol/kg) (B) and acetic acid (C) (mmol/kg) determined in doughs from legume flours processed by technological process 4.1 and fermentation process 4.2(sF and sgF). The fermentations were carried out using Lactobacillus plantarum DSM 33412 and Lactobacillus brevis DSM 33413 as starters, at 30° C. for 24 hours. The data are the result of three independent experiments (n=3). The top and bottom of the box represent the 75th and 25th percentiles of the data, respectively. The top and bottom of the bars represent the 5th and 95th percentiles of the data, respectively. The horizontal bar indicates the median of the distribution.

### DETAILED DESCRIPTION OF THE INVENTION

Fermentation of Pre-Gelatinised Flours with Selected Starters

The use of two selected strains of lactic bacteria was found to overcome the problems outlined above, resulting in gluten-free material with improved nutritional characteristics.

It is therefore an object of the present invention a fermentation process using such strains, as described below.

Said process is applied to gluten-free flowing material as defined above.

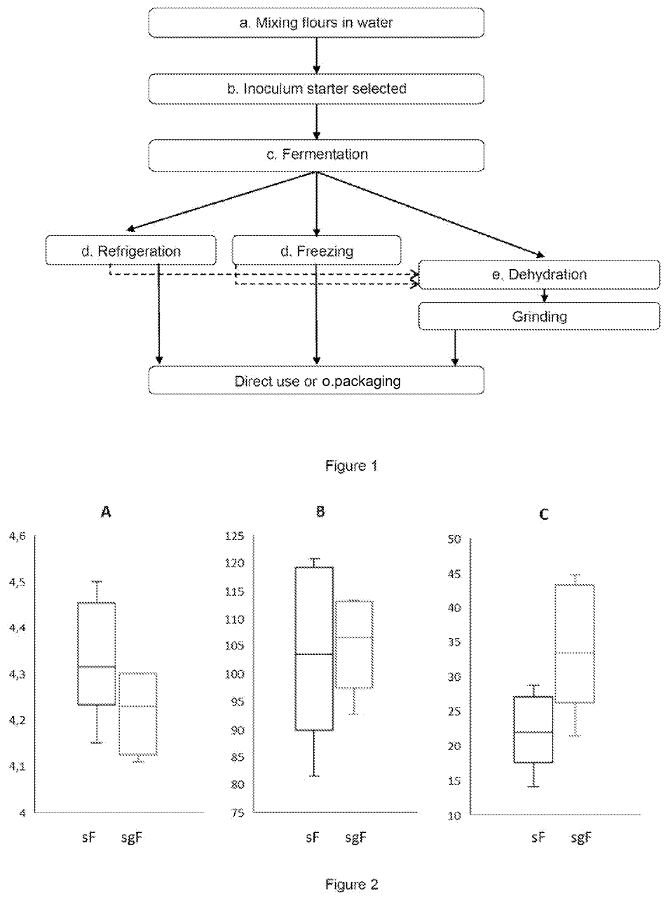

The features and advantages of the invention will best be apparent from the detailed description of a preferred example embodiment thereof, illustrated by way of non-limiting example with reference to the accompanying drawings in which FIG. 1 is a flow chart showing the various stages of the method of the invention.

Thus, an object of the invention is a strain of Lactobacillus plantarum (F.1) deposited by Favero Antonio S.r.l. with the Deutsche Sammlung von Mikroorganismen und Zellkulturen GmbH (German Collection of Microorganisms and Cell Cultures) on the date of 22 Jan. 2020 and which is identified by the deposit number DSM 33412.

This strain of Lactobacillus Plantarum DSM 33412 comprises at least one lactic acid bacterium isolated from spontaneously fermented chickpeas. It is a GRAM+, anaerobic bacterium. It can be grown in MRS medium (De Man, Rogosa, Sharpe) under the following conditions: incubation temperature 30° C., incubation time 24 hours. The pH of the medium is preferably about 6.1-6.2.

An object of the invention is also a strain of Lactobacillus brevis (F.4) which was deposited by Favero Antonio S.r.l. with the culture collection Deutsche Sammlung von Mikroorganismen und Zellkulturen GmbH (German Collection of Microorganisms and Cell Cultures) on the date of 22 Jan. 2020 and which is identified by the deposit number DSM 33413.

This strain of Lactobacillus brevis DSM 33413 comprises at least one lactic acid bacterium isolated from spontaneously fermented chickpeas. It is a GRAM+, anaerobic bacterium. It can be grown in MRS medium (De Man, Rogosa, Sharpe) under the following conditions: incubation temperature 30° C., incubation time 24 hours. The pH of the medium is preferably about 6.1-6.2.

Said strains can be left in microaerophilic conditions at about 16° C.-25° C. for up to 7 days. The strains of the present invention can be stored by means of methods known in the art for storing Lactobacillus strains. For example, they can be stored at −20° C. in an admixture with 20% v/v glycerol. The viability of the strains can also be assessed according to what is known in the sector, e.g., by placing them in the above-mentioned medium and checking the growth thereof after 24 hours.

A composition comprising said strain of Lactobacillus plantarum DSM 33412 is also an object of the present invention.

A composition comprising said strain of Lactobacillus brevis DSM 33413 is also an object of the present invention.

A composition comprising said strain of Lactobacillus plantarum DSM 33412 and said strain of Lactobacillus brevis DSM 33413, in any ratio, is also an object of the present invention.

The strains of the invention exhibit characteristics that make them particularly efficient under the application conditions of interest in the present invention and better than microorganisms known as starters for fermenting legume matrices.

In particular, these strains have a higher acidifying activity, a shorter latency phase and a higher acidification rate, with uniform performance in different matrices, compared to other known bacterial strains. Compared to the latter, they also have a greater ability to degrade anti-nutritional compounds that are present in the matrix, and to improve protein digestibility and numerous other nutritional aspects.

As mentioned above, said strains are advantageously used in a procedure for the fermentation of gluten-free flowing material.

The process for fermenting gluten-free flowing material according to the present invention comprises the following steps:
a) mixing the flowing material with drinking water. The weight/volume percentage of said flowing material in the admixture is in the range 30-60%, e.g., it is in the range 48-52%.
b) inoculating into the admixture thus obtained of an admixture of lactic bacteria comprising at least one strain selected from Lactobacillus plantarum DSM 33412 and Lactobacillus brevis DSM 33413.
c) fermenting the admixture at a temperature between 20 and 35° C., for example at 30° C., for a time between 8 and 24 hours until reaching a pH between 4.0 and 5.0, for example in the range 4.3-4.5, and a final cell density of the microorganisms in the range from 1 to 7×10⁹ cfu/ml;
d) optional refrigeration or freezing of the dough;
e) optional dehydration of the dough obtained at the end of each step c) or d);
f) optional packaging in material suitable for foodstuffs.

The fermentation process of the invention is carried out on gluten-free flowing material.

In particular, the fermentation process can be carried out on one or more of the following materials:
Legumes, such as, for example, bean, Phaseolus vulgaris L.; pea, Pisum sativum L.; fava bean, Vicia faba L.; lupin, Lupinus albus; chickpea, Cicer arietinum L.; pigeon pea, Cajanus indicus; groundnuts, Arachis hypogaea L.; soya, Glycine max; lenticchia, Lens culinaris; chickling vetch, Lathyrus sativus; carob tree, Ceratonia siliqua. Preferably, the legume is Cicer arietinum L. or Lens culinaris.
Pseudocereals, such as amaranth, Amaranthus spp.; quinoa, Chenopodium quinoa; buckwheat, Fagopyrum esculentum.
cereals which naturally do not have gluten, such as rice, maize, millet, teff, sorghum.

In one embodiment, the starting material is a legume having the following composition per 100 g of product: protein from 5 to 35 g, preferably 20 g, carbohydrate from 5 to 70 g, preferably 60 g, fibre from 2 to 20 g, preferably 10 g, fat from 1 to 50 g, preferably 5 g.

In one embodiment, the starting material is a cereal having the following composition per 100 g of product: protein from 5 to 15 g, preferably 8 g, carbohydrate from 30 to 90 g, preferably 70 g, fibre from 2 to 15 g, preferably 3, fat from 0.3 to 8 g, preferably 1.5 g.

The flowing material subjected to the process of the invention may also comprise an admixture of the above-mentioned materials, for example an admixture of legumes and cereals and/or pseudocereals.

The steps of the invention process will now be described in more detail and with reference to particular achievements of the invention.

In step a) the mixing the flowing material with drinking water can be carried out by any means suitable for the purpose and known to the expert in the field. Typically, mixing is carried out mechanically with stirrers, plunging arm mixers or planetary mixers.

In step b), an admixture of lactic bacteria comprising at least one of the two strains covered by the invention is inoculated into the material obtained at the end of step a).

In a preferred embodiment, the admixture of lactic bacteria comprises both strains Lactobacillus plantarum DSM 33412 and Lactobacillus brevis DSM 33413. In said embodiment, the two strains are present in a ratio Lactobacillus plantarum DSM 33412/Lactobacillus brevis DSM 33413 between 1:1 and 10:1, preferably 1:1.

The admixture of lactic bacteria may also comprise one or more bacteria belonging to one or more species selected from the group consisting of: Lactobacillus plantarum, Lactobacillus brevis, Lactobacillus rossiae, Lactobacillus sanfranciscensis, Pediococcus pentosaceus, Leuconostoc spp.

The bacteria are inoculated in a live, viable form in a liquid, pellet or lyophilised preparation.

The bacteria are inoculated so as to achieve a cell density preferably between 1 and 5×10⁷ cfu/ml of matrix to be fermented. For inoculation, the matrix can be brought to a temperature between 20 and 35° C., for example 30° C.

In step c) the material is fermented as described above.

The following steps are optional.

In optional step d) the dough obtained at the end of step c) is refrigerated at a temperature of between 4 and 12° C., e.g., 12° C., for a time of between 15 and 60 minutes, e.g. 30 min. This is advantageous since the chosen refrigeration temperature allows the admixture to be used for a period of 48 hours following the end of step c).

Alternatively, the dough obtained in step c) can be frozen at temperatures of −20° C. or lower for a time between 3 and 10 hours, e.g., 4 hours. Continuous freezing allows the admixture to be stabilised and used for a time after step c) of up to 6 months.

In the optional step e), which may follow any of steps c) or d), the material is dehydrated. Dehydration can be carried out e.g., by lyophilization or at low temperature, typically a temperature less than or equal to 70° C.

Preferably, in the optional step e), which may follow any of steps c) or d), the material is dehydrated at a temperature less than or equal to 65° C.

After dehydration, grinding can be carried out in order to obtain a material ground into a flour with a fine granulometry.

Dehydration allows the flour to be stabilised and used for up to 12 months after step c).

The material obtained at the end of each of the steps c), d) or e) can be used as an ingredient for the production of foodstuffs such as dough or oven-baked leavened products or breakfast extrudates.

In an optional step f) the obtained material is packed in suitable package for foodstuffs. Advantageously, such package is impermeable plastic if the material is packaged at the end of step c) or d) or paper if the material is packaged after step e).

The flour obtained by means of the method described above has the following advantageous characteristics:
gluten- and lactose-free;
low content or absence of anti-nutritional compounds: raffinose (and other alpha-galactosides), condensed tannins, phytic acid, trypsin inhibitors and saponins. In this regard, see examples 1 and 3.
contains lactic bacteria recognised as QPS (Qualified Presumption of Safety) by EFSA (Ricci et al., 2017. EFSA Journal, 15:1-178). Lactic acid bacteria are present in viable form and at high cell density, are present at a cell density>10⁹ cfu/g at the end of step c) or d) and are present at a cell density>10⁸ cfu/g at the end of step e). The obtained flour can therefore be used as a natural acidifying agent analogous to sourdough/“type II natural yeast” produced from gluten matrices. In this regard, see example 4;
maintains the concentration of fibre and protein of high biological value that characterise the unprocessed matrix of origin unaltered. See examples 1 and 3;
a lower glycaemic index than the unprocessed matrix of origin, thanks to the biological acidification operated by selected lactic bacteria and the increase in the percentage of resistant starch. See examples 1 and 3;
a high concentration of free amino acids. See examples 1 and 3;
a high digestibility of proteins, that is equal to or greater than 80%, thanks to the fermentation process operated with the selected lactic bacteria and the activation of proteolytic enzymes during the first steps of the technological process. See examples 1 and 3.

According to the invention, a flour obtained by the above-described method is therefore provided containing lactic bacteria in a viable form selected from a group comprising Lactobacillus plantarum, Lactobacillus brevis, Lactobacillus rossiae, Lactobacillus sanfranciscensis, Pediococcus and having a lactic bacteria cell density>10⁹ cfu/g at the end of step c) or d) and a cell density>10⁸ cfu/g at the end of step e).

Preferably, the flour containing lactic bacteria selected from, Leuconostoc spp. Lactobacillus plantarum DSM 33412 and Lactobacillus brevis DSM 33413.

According to the invention, a gluten-free flour obtained by means of the above-described method is therefore provided having at least some of the following characteristics:
protein digestibility>80%
starch hydrolysis index<64%
total free amino acid concentration>1.4 g/kg
concentration of resistant starch>2.5%
raffinose concentration<2 g/kg
phytic acid concentration<1.85 g/100 g
concentration of condensed tannins<2.9 mg/g.

Further details of these characteristics are shown in examples 1 and 3. These characteristics are obtained irrespective of the starting matrix used, i.e., the gluten-free starting material used, whether legume, cereal, pseudo-cereal or their admixture. In each case, the fermentation of the gluten-free material leads to obtaining a product characterized by at least some of the characteristics listed above.

In an advantageous version, the fermentation of the gluten-free material leads to obtaining a product characterized by all the characteristics listed above.

The obtained flour product therefore has a higher digestibility than corresponding flours obtained by known methods.

The obtained flour product further has improved nutritional properties compared to corresponding flours obtained by known methods.

The fermentation with the strains L. plantarum DSM 33412 and L. brevis DSM 33413 is therefore an effective treatment for improving the nutritional profile and reducing anti-nutritional factors in legume flours or other gluten-free materials.

No processes are known which would enable obtaining results of the same magnitude as those obtainable with the fermentation protocol described in the present invention.

Using the process of the invention, significant increases (P<0.05) were observed in: total polyphenol concentration and antioxidant activity, free amino acid concentration, protein digestibility and resistant starch concentration.

In one embodiment, the fermentation process described above can be used in combination with a technological pre-gelatinisation process.

Said pre-gelatinisation process is described in Italian patent application No. 102016000037027.

It is therefore also an object of the invention a method of processing gluten-free granular food material comprising the following steps:
providing a desired quantity of granular food material without gluten;
a step of humidification of said granular material, wherein said granular material is humidified in order to obtain a granular material having a humidity (UG) between 10 and 20%, for example between 16 and 18%;
a step of heating in order to heat the granular material up to a heating temperature (TH) between about 60 and about 100° C. by means of saturated vapour;
a feeding step for transferring said granular material from said heating step to a lamination step wherein said granular material is laminated at a lamination pressure (PL) of between 50-150 bar in order to obtain flowing material in the form of flakes;
a drying step, in which the flake-like flowing material is dried by means of hot air at a drying temperature (TE) between 140 and 160° C. in order to obtain flowing material at a humidity (UE) between 5 and 20%, for example between 10-15%,
a cooling step in which the flake-like flowing material is cooled to a cooling temperature (TC) between 5 and 15° C., wherein said cooling step is adjusted so that the flakes are cooled within a cooling time (tC) less than 60 sec, for example less than 30 sec;
a step of mixing said flowing material with drinking water, in which the material is present in the admixture at a weight/volume percentage in the range from 30 to 60%, for example 48-52%;
a step of inoculating an admixture of lactic bacteria comprising at least one strain selected from the strain of Lactobacillus plantarum according to claim 1 and the strain Lactobacillus brevis according to claim 2;
a step of fermenting the admixture at a temperature between 20 and 35° C., for example at 30° C., for a time between 8 and 24 hours until reaching a pH between 4.0 and 5.0, for example in the range 4.3-4.5, and a final cell density of the microorganisms in the range from 1 to 7×10⁹ cfu/ml;
a subsequent optional step of refrigeration, at a temperature between 4 and 12° C., or freezing, at temperatures less than or equal to −20° C.;
an optional step of dehydration, following the step of fermentation or the step of refrigeration, which may optionally be followed by grinding in order to obtain a flour with a fine granulometry;
an optional packaging stage, which may be subsequent to any of the preceding fermentation, refrigeration, freezing or dehydration stages in which the obtained material is packaged in suitable package for foodstuffs.

Further details relating to the pre-gelatinisation process, i.e., the process which in the embodiment described above precedes the mixing of the flowing material with water and its subsequent fermentation, are described in patent application No. 102016000037027, to which reference may be made.

The sequential combination of gelatinisation processing with fermentation under controlled conditions allows a further reduction of anti-nutritional compounds, compared to that obtained with the fermentation process alone. There are significant further decreases in raffinose and condensed tannin levels, with complete degradation of trypsin inhibitors, phytic acid and saponins.

Compared to pre-gelatinised flours at the end of the technological process, the fermentation leads to an increase in total polyphenols and antioxidant activity, free amino acid concentration, protein digestibility and resistant starch compared to unfermented flours.

Subjecting a gluten-free material to a pre-gelatinisation and fermentation process results in a flour product with at least some of the following characteristics:
protein digestibility>99%
starch hydrolysis index<50%
total free amino acid concentration>1.9 g/kg
concentration of resistant starch>8%
raffinose concentration<0.6 g/kg
absence of phytic acid (0%)
concentration of condensed tannins<1.8 mg/g

Further details are evident from examples 1 and 3.

Advantageously, subjecting a gluten-free material to a pre-gelatinisation and fermentation process results in a flour product with all the characteristics mentioned above.

At least some of the above characteristics are obtained irrespective of the starting matrix used, i.e., the gluten-free starting material used, whether legume, cereal, pseudo-cereal or admixture thereof. In each case, the fermentation of the gluten-free material leads to obtaining a product characterized by at least some of the characteristics listed above.

The obtained flour product therefore has a higher digestibility than corresponding flours obtained by known methods.

The obtained flour product further has improved nutritional properties compared to corresponding flours obtained by known methods.

No processes are known which would enable obtaining results of the same magnitude as those obtainable with the fermentation protocol described in the present invention.

Furthermore, the flowing material obtained by means of the method of the invention can be used, also in frozen and dehydrated form, as a biological fermentation and acidification agent by virtue of the high cell density of lactic bacteria that characterises it, given the high survival of the selected strains Lactobacillus plantarum DSM 33412 and Lactobacillus brevis DSM 33413 to the physical stabilisation processes of the processed matrix.

There are no known products among the gluten-free produced experimental food flours nor commercial products in which the combination of the nutritional and microbiological characteristics described is demonstrable.

The flowing material obtained by means of the method of the invention can advantageously be used to make gluten-free flours, flour mixtures from different legumes, cereals or cereal-like grains, oven-baked goods, pasta, snacks or other.

In particular, oven-baked goods, pasta, snacks or the like can be made starting from the material obtained with the method of the present invention using the common methods known in the art for producing oven-baked products, pasta and the like from a flour.

The material obtained with the method of the invention can also be used as an acidifying agent in the production of oven-baked goods.

The present invention will now be illustrated by means of examples.

### EXAMPLES

### Example 1

Nutritional Characterisation of Pre-Gelatinised Flours (Technological Processing) in Comparison with Unprocessed Flours

Materials and Methods

Raw Materials

Yellow and red lentil, white and black bean, chickpea and pea were fully ground and analysed (s:—“flours”, unprocessed). The same batches of legumes were subjected to the technological process subject matter of the present invention to obtain pre-gelatinised flours (sg—“gelatinised flours”).

Protein (total nitrogen×5.7), lipids, ash and humidity of unprocessed and processed flours were determined according to the methods approved by AACC 46-11A, 30-10.01, 08-01 and 44-15A, respectively (American Association for Clinical Chemistry AACC, 2010). The carbohydrates were determined by difference.

The microbiological analyses were carried out on 10 g of flour, homogenised with 90 ml of sterile water containing 0.9% [w/v] NaCl). The lactic bacteria (LAB) were enumerated by plate counts on De Man, Rogosa and Sharpe (MRS) (Oxoid, Basingstoke, Hampshire, UK) agar, supplemented with cycloheximide (0.1 g/l). The plates were incubated under anaerobic conditions (AnaeroGen and AnaeroJar, Oxoid) at 30° C. for 48 hours. The cell density of yeasts and moulds was estimated by counts on Agar Yeast Peptone Dextrose (YPDA) medium (Sigma-Merck, Darmstadt, Germany) supplemented with chloramphenicol (0.1 g/l), and enumerated by inclusion and spreading, respectively, and the plates were incubated at 30° C. for 72 hours. The identification of yeasts or moulds was carried out by visual analysis of colony morphology. Enterobacteriaceae were determined on Violet Red Bile Glucose Agar (VRBGA, Oxoid) at 37° C. for 24 hours.

Organic Acids, Fermentation Quotient and Free Amino Acids

Water/salt-soluble extracts (WSE) used to analyse the concentrations of free amino acids (FAA) and organic acids from legume flours unprocessed and processed by technological process 4.1 were prepared according to the method originally described by Weiss et al. (Weiss, et al., 1993. Electrophoresis, 14, 805-816.3). Fifteen g of sample was suspended in 60 ml Tris-HCl (50 mM, pH 8.8), kept at 4° C. for 1 hour, vortexed at 15-minute intervals, and centrifuged at 20000 g for 20 minutes. The supernatant was used for analysis. FAAs were analysed by means of an amino acid analyser, Biochrom 30 series (Biochrom Ltd., Cambridge Science Park, England) with a cation exchange column (Na; 20×0.46 cm internal diameter), as described by Rizzello et al. (Rizzello et al., 2010. Food Chemistry, 119: 1079-1089).

The determination of organic acids in the soluble extract was carried out by HPLC liquid chromatography, using an ÄKTA Purifier system (GE Healthcare, Buckinghmshire, UK), provided with an Aminex HPX-87H column (Molecular Exclusion, Biorad, Richmond, Calif.), and a 210 nm UV detector. The elution was carried out at 60° C., with a flow rate of 0.6 ml/min, using H₂SO₄ (10 mM) as the mobile phase.

The fermentation quotient (QF) was determined as the molar ratio between lactic and acetic acid.

Dietary Fibre and Resistant Starch

Insoluble dietary fibre (IDF) and soluble fibre (SDF) were determined according to the procedure previously described by Goni et al. (Goñi et al., 2009. Food Research International, 42:840-846) for solid samples. Specifically, 300 mg of each sample was weighed into 50 ml centrifuge tubes and resuspended in 10 ml of phosphate buffer and 0.2 ml of pepsin solution (2000 U/g). The samples were incubated at 40° C. for 1 hour. Pancreatin (5 mg/ml) was added to the sample mixtures and the pH was adjusted to 7.5. The samples were incubated at 37° C. for 6 hours. Subsequently, amylase (110 U/ml) was added and the samples (pH corrected to 6.9) were incubated at 37° C. for 16 hours under stirring. The samples were then centrifuged (15 min, 3000×g) and the supernatants were discarded and collected. The pellets were washed twice with 5 ml of distilled water while all supernatants were collected and mixed. The pellets were dried overnight at 105° C., cooled and weighed to determine the weight of the residue, corresponding to the IDF fibre. A sodium acetate buffer (pH 4.75) was added to the supernatants, followed by 0.1 ml amyloglucosidase (140 U/ml), and then incubated at 60° C., for 45 min, under stirring. The mixtures were transferred to dialysis membranes (cut-off 14000 Da) and dialysed against water at 37° C. for 48 h. The dialysates were is lyophilized. The residues, corresponding to the SDF fibre, were quantified gravimetrically.

The resistant starch of the flours was determined according to the method AACC 32-40.01.01 3 (AACC, 2010).

Total Polyphenols and Antioxidant Activity

The total polyphenols were determined on the methanolic extract (ME) of the flours. Five grams of each sample was mixed with 50 ml of 80% methanol to obtain ME. The admixture was bubbled with nitrogen for 30 min, under stirring conditions, and centrifuged at 4600×g for 20 min. The methanolic extracts were transferred into tubes, subjected to nitrogen flux again and stored at about 4° C. prior to analysis. The method used to determine the antioxidant activity is a spectrophotometric method which envisages the use of butylated hydroxytoluene (BHT), a synthetic alkylated phenol, as a reference. The protocol involves the sample preparation using 167 μl of ME, 167 μl of 80% methanol solution containing DPPH (final concentration in the reaction admixture 100 μM) and 667 μl of solvent (80% v/v methanol). A negative control was obtained using 167 μl of DPPH solution and 833 μl of solvent while a positive control was analysed using BHT at a concentration of 75 ppm and processed as a sample. The spectrophotometric readings were taken immediately following mixing of the solutions at a wavelength (A) equal to 517 nm (against air) and repeated every two minutes for thirty minutes. The scavenging activity towards DPPH was calculated using the following formula:

antioxidant activity (%)=[(blank absorbance−sample absorbance)/blank absorbance]*100.

The concentration in total polyphenols was determined as described by Slinkard and Singleton (Slinkard and Singleton, 1977. American Journal of Enology and Viticulture, 28:49-55) and expressed as gallic acid equivalent. Also in this case, the analysis is carried out on MEs (obtained as described above). 1.58 ml of distilled water, 100 μl of Folin-Ciocalteu reagent (Sigma), 20 μl of ME were introduced into the cuvettes and mixed thoroughly. After waiting between 30 sec and 8 min, 300 μl of a saturated sodium carbonate solution was added. The cuvettes were incubated at 20° C. for 2 hours and finally a spectrophotometric reading was taken at a A of 765 nm. The calibration straight line was obtained with standard solutions of known concentration of gallic acid (0, 50, 100, 150, 250, and 500 mg/L), subsequently processed as samples. The concentration of phenols was expressed as mg/kg of dough.

Anti-Nutritional Factors

The flours were analysed for raffinose content, condensed tannins, trypsin inhibitors, phytic acid (and phytasic activity), and saponins. The concentration of phytic acid and raffinose was measured using the Megazyme Kit K-PHYT 05/07 and the Raffinose/D-Galactose Assay Kit K-RAFGA (Megazyme International Ireland Limited, Bray, Ireland), following the manufacturer's instructions.

The phytasic activity was determined on the WSEs of processed and unprocessed legume flours by monitoring the rate of hydrolysis of p-nitrophenyl phosphate (p-NPP) (Sigma, 104-0). The reaction admixture contained 200 μl of 1.5 mM p-NPP (final concentration) in Na-acetate (0.2 M, pH 5.2), and 400 μl of WSE. The admixture was incubated at 45° C. and the reaction was stopped by adding 600 μl of NaOH (0.1 M). The released p-nitrophenol was determined by measuring the absorbance at 405 nm (Rizzello, et al., 2010. Food Chemistry, 119:1079-1089). A unit (U) of activity was defined as the amount of enzyme required to liberate 1 μmol/min of p-nitrophenol under the test conditions.

The condensed tannins were determined using the test described by Hagerman (Hagerman, 2002. In: Hagerman (Ed.), The Tannin Handbook. Miami University, Oxford). The sample preparation involved the use of 1 ml of ME to which 6 ml of iso-butanol were added, prepared by mixing 950 ml of n-butanol with 50 ml of concentrated HCl. Subsequently, 0.2 ml of ferric ions were introduced, obtained by using ferric ammonium sulphate at 2% in HCl (2 N) and stored in the dark. The admixture containing the sample was homogenised by vortexing and placed in a thermostatic bath at 100° C. for 50 minutes. Once the sample was cooled, the absorbance at 550 nm was read by subtracting the absorbance of the blank containing only the sample solvent (methanol), butanol acid and ferric ions from the sample absorbance. The quantification of the tannins was carried out by relating the absorbance value obtained from the different samples to the calibration straight line obtained using scalar concentrations of a compound belonging to the proanthocyanidins family, specifically from sorghum, as a standard.

The trypsin inhibitors were determined as described by Alonso et al. (Alonso, et al., 2000. Food Chemistry, 68:159-165), using α-N-benzoyl-DL-arginine-p-nitroanilidehydrochloride (BApNA) as a substrate for trypsin. The trypsin inhibitory activity (TIA), expressed as units of inhibition (U) on mg sample, was calculated from the absorbance read at 410 nm against the blank. One unit was calculated as a 0.01 unit absorbance increase at 410 nm of the reaction admixture.

The total saponins in the flours were determined as reported by Lai et al. (Lai et al., 2013. Journal of Bioscience and Bioengineering, 115(5), 552-556) with some modifications. Briefly, the lyophilized samples (0.5 g) were mixed with 10 ml petroleum ether (Carlo Erba) by stirring for 4 hours. The residues (20 mg) were then extracted with 5 ml of methanol 80% (v/v) with stirring for 4 hours. The extracts were kept at 4° C. in the dark until they were analysed. The total saponin content (TSC) was determined by the spectrophotometric method with the reading at 554 nm against the blank. All data are expressed on a dry weight basis.

In Vitro Protein Digestibility

In vitro protein digestibility (IVPD) was determined using the method proposed by Akeson and Stahmann (Akeson and Stahmann, 1964. Journal of Nutrition, 83 (2), 257-261) with some modifications (Rizzello C., et al., 2016. Food Microbiology, 56,1-13). Samples were subjected to sequential enzymatic treatment modelled on in vivo digestion in the gastrointestinal tract. One gram of each sample was incubated with 1.5 mg pepsin, in 15 ml HCl (0.1 M), at 37° C. for 3 hours. After neutralisation with NaOH (2 M) and the addition of 4 mg of pancreatin in 7.5 ml of phosphate buffer (pH 8.0), 1 ml of toluene was added to prevent microbial growth. The solution was incubated for 24 hours at 37° C. After 24 hours, the enzyme was inactivated by the addition of 10 ml of trichloroacetic acid (20%, w/v) and to thus allow the precipitation of the undigested protein. The volume was brought to 100 ml with distilled water and the admixture centrifuged at 5000 g for 20 minutes. IVPD was expressed as the percentage of total protein that was solubilised after enzymatic hydrolysis. The protein concentration of the digested and undigested fractions was determined by the Bradford method (Bradford, 1976. Analytical Biochemistry, 72, 248-54).

Starch Hydrolysis

The starch hydrolysis analysis was performed on processed and unprocessed flours. The in vitro procedure mimics the in vivo digestion of the starch (De Angelis, et al., 2009. European Food Research and Technology, 229, 593-601). Aliquots of flours, containing 1 g starch, were subjected to enzymatic digestion process and the glucose content released was measured using the D-Fructose/D-Glucose Assay Kit (Megazyme). The degree of starch digestion was expressed as the percentage of potentially available starch hydrolysed after 180 min. A control consisting of wheat bread (WB) leavened with brewer's yeast was used to estimate the hydrolysis index (HI=100).

Statistical Analyses

All biochemical analysis data were obtained at least in duplicate and each replicate was analysed twice. The data were subjected to one-way ANOVA using IBM SPSS Statistics 26 software (IBM Corporation, New York City, N.Y., USA).

Results

Chemical and Microbiological Analysis of Flours Processed and Unprocessed with the Process of the Invention

The composition of the flours is shown in Table 1 below.

TABLE 1
Average nutritional values (%) of legume flours.

| | | Yellow | White | Black | | |
|---|---|---|---|---|---|---|
| | Red lentil | lentil | bean | bean | Chickpea | Pea |
| Humidity (%) | 10 ± 0.3 | 7.8 ± 0.3 | 8.0 ± 0.1 | 8.2 ± 0.3 | 9.4 ± 0.2 | 10.6 ± 01 |
| Proteins (%) | 24.5 ± 0.2 | 26.0 ± 0.3 | 23.7 ± 0.5 | 19.2 ± 0.1 | 19.0 ± 0.3 | 19.7 ± 1.3 |
| Fat (%) | 1.9 ± 0.2 | 1.7 ± 0.2 | 2.3 ± 0.2 | 3.6 ± 0.1 | 4.1 ± 0.2 | 2.3 ± 0.2 |
| Carbohydrates | 61.0 ± 1.2 | 62.5 ± 1.3 | 63.0 ± 1.3 | 65.1 ± 1.4 | 63.8 ± 1.3 | 64.4 ± 1.3 |
| (%) | | | | | | |
| Ashes (%) | 2.5 ± 0.1 | 2.1 ± 0.2 | 3.0 ± 0.1 | 4.1 ± 0.3 | 3.7 ± 0.3 | 3.0 ± 0.2 |

The data are the result of three independent experiments ± standard deviation (n = 3).
The results for protein, fat, carbohydrates and ash are given as % of dry matter.
The humidity was between 7 and 11% for all flours s.

The protein content found in the red and yellow lentil and white bean flours was in the range of 23.7±0.5-26.0±0.3% (dry matter), while significantly lower values (P<0.05) were found in the black bean, chickpea and pea flours (ca. 19% dry matter). The fat content was 3.6% lower in all flours s, with the exception of chickpea flour, which contained a significantly (P<0.05) higher amount (4.1±0.2). The ash content was lower by about 3.0% (dry matter), except for black bean and chickpea flours (Table 1).

The results of the microbiological analyses are shown in Table 2.

All microbial groups investigated were detected in the samples analysed at a lower density of about 4 log 10 cfu/g. White bean flour contained the highest cell density of lactic bacteria and yeasts, while moulds and Enterobacteriaceae were more abundant in red lentil and black bean flours (Table 2).

The technological treatment mainly influenced the cell density of yeasts and Enterobacteriaceae. In fact, a significant (P<0.05) decrease was found in all the flours (Table 2). The greatest decrease in yeasts and Enterobacteriaceae, equal to about 2 log 10 cfu/g was found in white and black bean flours. LAB and moulds remained almost constant during the technological processing in all the flours (Table 2).

TABLE 2
Microbiological characterisation (Log cfu/g) of unprocessed legume flours (s) and processed by the technological
process of the invention (sg).

| | s | | | | | | sg | | | | | |
|---|---|---|---|---|---|---|---|---|---|---|---|---|
| | Red | Yellow | White | Black | | | Red | Yellow | White | Black | | |
| | Lentil | Lentil | Bean | Bean | Chickpea | Pea | Lentil | Lentil | Bean | Bean | Chickpea | Pea |
| LAB* | 2.7 ± 0.2^{a,c} | 2.3 ± 0.4^{c} | 3.5 ± 0.2^{a} | 3.2 ± | 2.7 ± 0.3^{c} | 3.6 ± | 3.2 ± 0.1^{b} | 2.6 ± 0.2^{c} | 3.7 ± 0.3^{a} | 3.1 ± 0.2^{c} | 2.4 ± 0.2^{c} | 3.6 ± |
| | | | | 0.3^{b} | | 0.1^{a,b} | | | | | | 0.2^{e} |
| Yeasts | 3.5 ± 0.3^{b} | 1.5 ± 0.3^{e} | 4.2 ± 0.3^{a} | 3.6 ± | 2.1 ± 0.2^{d} | 4.2 ± 0.2^{a} | 1.8 ± 0.2^{d} | 1.2 ± 0.3^{c} | 1.8 ± 0.2^{d} | 2.4 ± 0.3^{c} | 1.2 ± 0.3^{e} | 2.1 ± |
| | | | | 0.4^{a,b} | | | | | | | | 0.1^{d} |
| Moulds | 3.5 ± 0.3^{a} | 2.1 ± 0.2^{d} | 2.4 ± 0.1^{c} | 2.9 ± 0.3^{b} | 2.3 ± 0.2^{d} | 1.6 ± 0.1^{f} | 3.5 ± 0.2^{a} | 1.8 ± 0.2^{e} | 2.7 ± 0.1^{c} | 2.7 ± 0.2^{b} | 2.4 ± 0.4^{d} | 1.6 ± |
| | | | | | | | | | | | | 0.4^{e} |
| Entero- | 3.3 ± 0.2^{b,c} | 2.8 ± 0.2^{c} | 3.5 ± 0.3^{b} | 4.1 ± 0.2^{a} | 2.4 ± 0.3^{d} | 4.3 ± 0.3^{a} | 2.1 ± 0.3^{d,e} | 1.7 ± 0.2^{e} | 1.4 ± 0.1^{f} | 2.2 ± 0.4^{d,e} | 1.6 ± 0.3^{f} | 2.3 ± |
| bacteriae | | | | | | | | | | | | 0.3^{d} |
| cea | | | | | | | | | | | | |

LAB, lactic bacteria.
The data are the result of three independent experiments ± standard deviation (n = 3).
^{a-f}Values in the same row with different superscript letters are significantly different (P <0.05).

In addition, no organic acids, such as lactic acid and acetic acid, were detected in the flours s and sg.

Total Polyphenols and Antioxidant Activity

The quantification of the total polyphenols and the evaluation of the detoxification activity against the synthetic radical DPPH were carried out on MEs of the flours s and sg.

Red lentil and chickpea flours had the lowest and highest concentrations of total polyphenols, respectively (Table 3).

Overall, the sg flours showed a slightly lower total phenol content (21%) than the corresponding s flours, with the exception of red lentil flour, which showed a similar value before and after technological processing (P>0.05). Antioxidant activity followed the same trend (Table 3). In fact, the values of the scavenging activity determined on DPPH were up to 30% lower in sg than in s, while it was over 38% in all sg with the exception of chickpea, characterised by a lower value. The highest activity was found in black bean, both s and sg (Table 3).

Proteolysis and IVPD

The degree of proteolysis was studied on all flours, processed and unprocessed, by determining the concentration of the free amino acids (TFFA).

In detail, thermal processing significantly resulted in an increase in TFAAs (Table 3). With the exception of yellow and red lentil flours, in which a slight and non-significant (P<0.05) increase was found, a higher TFAA concentration was found in the sg flours than in the corresponding s flours as a consequence of the processing (Table 3). The highest increase was observed in sg pea flours (ca. 42%), while the highest concentration was found in chickpea flour (2217±62 mg/kg).

The advantages deriving from the increase of free amino acids, released by hydrolysis from proteins, in a food, are manifold.

For example, from a nutritional point of view they are immediately bioavailable and can be assimilated in the intestine without undergoing the digestive degradation processes that proteins require.

From a sensory point of view, free amino acids have marked sensory attributes that are not possessed by proteins in their native status: they are, for example, responsible for the taste (particularly the sapidity) of the foodstuff containing them; furthermore, further reactions in amino acids, such as those occurring during baking, can lead to the formation of volatile compounds that enrich the olfactory profile of the food product.

The IVPD of the s flours ranged from 71±2 (red lentil) to ca. 79% (chickpea and white bean). Overall, the extent of the proteolysis was reflected in the IVPD. In fact, the technological processing 4.1 caused moderate increases (up to 9.5%) in IVPD values in sg compared to the corresponding s (Table 3). Among sg flours, the highest IVPD was found in white bean, followed by chickpea and black bean.

The IVPD parameter is a nutritional index of great importance in the field of nutrition: it indicates what percentage of the matrix protein is actually digested during gastro-intestinal transit as a result of the action of digestive enzymes. Only this aliquot, the digestible one, contributes positively to the consumer's nutritional status with its supply of assimilable essential and non-essential amino acids.

Increasing such parameter is therefore nutritionally important and is of particular value in vegetarian and vegan diets, where vegetable proteins alone, often because of their low digestibility as well as the composition thereof, do not fully meet the individual's need for essential amino acids.

Resistant Starch, Fibre and Starch Hydrolysis

The resistant Starch (RS) is the fraction of starch that resists to the hydrolysis process by digestive enzymes in vitro and in vivo. Because of these characteristics, some of its subtypes are considered part of the insoluble dietary fibre (Cabras and Martelli, 2004. Chimica degli alimenti). The technological process has influenced the resistant starch. In fact, the fraction of resistant starch was significantly higher in the sg flours than in the corresponding s flours (P<0.05). The highest concentration was found in black bean flours (9.17±0.086 and 11.66±0.02%, in s and sg, respectively) (Table 3). In addition, the technological process 4.1 did not cause significant changes in the concentration of SDF fibres (Table 4), while an increase (of about 16%) was found in the concentration of IDF in the sg flours compared to s flours (Table 4). With the only exception of red lentil and chickpea flours, pre-gelatinisation produced a slight increase in TDF concentration in all flours (Table 4). Overall, the values ranged from 21.2±0.6% to 29.1±0.6% and from 21.3±0.3% to 30.7±0.4% in s and sg, respectively. The highest TDF contents were found in sg chickpeas and black bean (>30%).

By not contributing to the release of glucose during the digestive process, the resistant starch and dietary fibres (TDF, IDF, SDF) do not lead to an increase in peak blood sugar levels after ingestion of starch or simple sugars.

The increase in TDF, IDF and SDF in pregelatinised flours therefore leads to a product characterised by a glycemic index, allowing to obtain nutritionally more balanced foodstuffs with a reduced calorie content in the diet. This can be seen in the starch hydrolysis index (HI), which has been influenced by the technological process. In fact, the sg flours had a slightly lower HI than the corresponding s flours (mean value of 57.3 vs. 50%, respectively) (Table 3). The starch hydrolysis index is an analytical parameter used as a predictor of the glycemic index of foodstuffs. Proportionally to the decrease in its value, it is possible to find a decrease in the glycemic index.

TABLE 3
Concentration of total polyphenols, antioxidant activity (determined on DPPH radical), total free amino acids (TFAA), in vitro protein
digestibility (IVPD), resistant starch and starch hydrolysis (HI) of unprocessed and processed flours by means of the technological process
of the invention (s and sg, respectively).

| | s | | | | | | sg | | | | | |
|---|---|---|---|---|---|---|---|---|---|---|---|---|
| | Red | Yellow | White | Black | Chick | | | Yellow | White | Black | | |
| | Lentil | Lentil | Bean | Bean | pea | Pea | Red Lentil | Lentil | Bean | Bean | Chickpea | Pea |
| Total | 0.45 ± | 0.53 ± | 0.52 ± | 0.66 ± | 1.17 ± | 0.86 ± | 0.43 ± 0.02^{f} | 0.42 ± 0.03^{f} | 0.32 ± | 0.53 ± 0 02 [symbol] | 1.04 ± | 0.67 ± |
| phenols | 0.01^{f} | 0.01^{a} | 0.01^{e} | 0.02^{d} | 0.03^{a} | 0.02^{c} | | | 0.01^{g} | | 0.01^{b} | 0.01^{d} |
| (mmol/kg) | | | | | | | | | | | | |
| DPPH (%) | 49.9 ± | 56.3 ± | 46.7 ± | 78.3 ± | 43.4 ± | 43.2 ± | 47. 9± 1.2^{e} | 51.1 ± 1.4^{d} | 38.9 ± | 72.0 ± 1.0^{b} | 31.5 ± 1.4^{h} | 38.7 + |
| | 2.4^{de} | 1.2^{c} | 2.3^{c} | 1.9^{a} | 2.1^{f} | 2.1^{f} | | | 1.7^{g} | | | 1.4^{g} |
| TFFA | 2001 ± | 1690 ± | 1697 ± | 1198 ± | 1968 ± | 1023 ± | 2078 ± 100^{b} | 1835 ± 84^{c} | 1982 ± | 1441 ± 75^{e} | 2217 ± 62^{a} | 1454 ± |
| (mg/kg) | 99^{b} | 78^{c} | 87^{d} | 68^{a} | 87^{b} | 59^{f} | | | 97^{b} | | | 77^{e} |
| IVPD (%) | 71 ± 2^{d} | 73 ± 1^{c} | 78 ± 2^{b} | 75 ± 3^{c} | 79 ± 3^{b} | 76 ± 5^{c} | 77 ± 3^{b} | 76 ± 3^{b} | 92 ± 5^{a} | 84 ± 3^{a} | 88 ± 2 | 78 ± 3^{b} |
| Resistant | 2.09 ± | 2.38 ± | 8.78 ± | 9.17 ± | 2.9 + | 2.5 ± | 2.75 ± 0.04 [symbol] | 2.84 ± 0.2^{f} | 9.57 ± | 11.66+0.02^{a} | 3.41 ± | 3.6 ± |
| starch (%) | 0.13^{gg} | 0.07^{gg} | 0.05^{c} | 0.06^{b} | 0.07^{f} | 0.08^{f} | | | 0.03^{b} | | 0.06^{e} | 0.05^{d} |
| HI (%) | 56 ± 3^{a} | 59 ± 3^{c} | 66 ± 1^{a} | 66 ± 3^{a} | 49 ± 2^{f} | 64 ± 3^{a,b} | 55 ± 3^{e} | 56 ± 1^{e} | 61 ± 2^{b} | 64 ± 3 [symbol] | 46 ± 2^{g} | 62 ± 1^{c} |

^{a-h}Values in the same row with different superscript letters are significantly different (P <0.05).
The data are the result of three independent experiments ± standard deviation (n = 3). [symbol]
indicates data missing or illegible when filed

TABLE 4
Soluble dietary fibres (SDF), insoluble dietary fibres (IDF) and total dietary fibres (TDF) in unprocessed and processed flours by means of
the technological process of the invention (s and sg, respectively). Within the analytical category (SDF, IDF and TDF), values labelled with different
letters differed significantly (P <0.05).

| | SDF | | IDF | | TDF | |
|---|---|---|---|---|---|---|
| | s | sg | s | sg | s | sg |
| Red lentil | 5.1 ± 0.6 [symbol] | 5.1 ± 0.4 [symbol] | 19.1 ± 0.6 [symbol] | 19.6 ± 0.4 [symbol] | 24.1 ± 0.5 [symbol] | 25.2 ± 0.8 [symbol] |
| Yellow lentil | 3.0 ± 0.4 [symbol] | 3.1 ± 0.2 [symbol] | 17.4 ± 0.7 [symbol] | 18.3 ± 0.6 [symbol] | 21.2 ± 0.6 [symbol] | 21.3 ± 0.3 [symbol] |
| White bean | 3.1 ± 0.4 [symbol] | 3.1 ± 0.1 [symbol] | 23.1 ± 0.5 [symbol] | 24.1 ± 0.4 [symbol] | 26.3 ± 0.6 [symbol] | 27.4 ± 0.5 [symbol] |
| Black bean | 5.1 ± 0.3 [symbol] | 5.1 ± 0.5 [symbol] | 23.12 ± 0.7 [symbol] | 25.3 ± 0.5 [symbol] | 27.1 ± 0.5 [symbol] | 30.7 ± 0.4 [symbol] |
| Chickpea | 8.3 ± 0.2 [symbol] | 8.2 ± 0.2 [symbol] | 20.4 ± 0.5 [symbol] | 22.1 ± 0.2 [symbol] | 29.1 ± 0.6 [symbol] | 30.3 ± 0.5 [symbol] |
| Pea | 8.1 ± 0.4 [symbol] | 8.3 ± 0.1 [symbol] | 16.5 ± 0.4 [symbol] | 19.1 ± 0.6 [symbol] | 25.2 ± 0.7 [symbol] | 27.4 ± 0.6 [symbol] |

[symbol]

indicates data missing or illegible when filed

Antinutritional Factors

Raffinose

The raffinose concentration in s was in the range 1.64±0.03 to 2.93±0.12 g/kg. The bean (white and black) and red lentil flours had the lowest and highest contents respectively (Table 5). The technological process resulted in a decrease of raffinose concentration in sg flours compared to s flours. In detail, reductions from 17.5 to 46% were found in sg. The largest decrease was found in black bean sg flour (Table 5). Lentil flours, both red and yellow, contained the highest concentration of raffinose, both before and after gelatinisation.

Condensed Tannins

The concentration of condensed tannins in s flours ranged from 0.71±0.04 to 3.02±0.05 mg/g; black and white bean flours contained the lowest and highest concentrations. The technological process has not affected its content. In fact, there were no significant differences between s and sg (Table 5).

Trypsin Inhibitor Activity

Trypsin inhibitor activity ranged from 0.43±0.02 to 0.85±0.03 U in the s flours. With the only exception of pea flour (0.43±0.02 U), all samples were characterised by similar TIA values (ca. 0.8 U) (Table 5). Following the technological process, the activity of trypsin inhibitors decreased from 22% to 56%. Pea flour had the greatest decrease and showed the lowest activity before and after gelatinisation (Table 5).

Phytic Acid and Phytasic Activity

The phytic acid ranged from 1.23±0.05 to 2.64±0.06 g/100 g in the s flours. Chickpea and pea flours contained the highest and lowest amounts of the compound, respectively. The phytic acid was sensitive to the technological processing, in fact sg flours were characterised by concentrations about 57% lower than s. Although the greatest decrease was found in red lentil and white bean flours, pea sg flour contained the lowest concentration (Table 5). The phytasic activity, as a result of the technological process, increased significantly: in detail, it increased about 2-fold compared to the corresponding s. The red lentil flours (s and sg) were characterised for the highest phytasic activity (Table 5).

Total Saponins

The total saponins ranged from 0.64±0.02-1.47±0.05 mg/g (Table 5). After technological processing, the concentration significantly decreased in all the flours. In fact, a general decrease was observed, from about 19 to 52% (Table 5).

### Concluding Remarks

Regardless of the type of legume processed, the technological process results in a reduction of some anti-nutritional compounds. In particular, compared to unprocessed flours, in pre-gelatinised flours it is observed that:
raffinose, a sugar that is not digestible by the human body but can be fermented by intestinal micro-organisms with the development of gas, decreases by 40-70%;
condensed tannins decrease by 2-13%;
the activity of trypsin inhibitors decreases by 20-50%;
phytic acid (a compound that can complex and make minerals and proteins unavailable) decreases by 30-60%;
saponins (capable of reducing protein digestibility) decrease by 20-50%.

The nutritional value of the processed samples is also positively influenced by the increase in free amino acid concentration and protein digestibility (4-40% and 3-18%, respectively).

TABLE 5
Raffinose, condensed tannins, phytic acid, saponins and trypsin and phytasic inhibition activity on unprocessed and processed flours by
means of the technological process of the invention (s and sg, respectively).

| | s | | | | | | sg | | | | | |
|---|---|---|---|---|---|---|---|---|---|---|---|---|
| | Red | Yellow | White | Black | | | | Yellow | White | Black | | |
| | Lentil | Lentil | Bean | Bean | Chickpea | Pea | Red Lentil | Lentil | Bean | Bean | Chickpea | Pea |
| Raffinose | 2.93 ± | 2.73 ± | 1.64 ± | 1.66 ± | 1.83 ± 0.08^{c} | 2.11 ± 0.12^{b} | 1.95 ± 0.07^{e} | 2.03 ± 0.12^{b,c} | 1.21 ± | 0.96 ± 0.12^{h} | 1.30 ± 0.06^{e,f} | 1.15 ± 0.03^{g} |
| (g/kg) | 0.12^{a} | 0.04^{a} | 0.03^{d} | 0.07^{d} | | | | | 0.05^{f} | | | |
| Condensed | 1.12 ± | 0.95 ± | 0.71 ± | 3.02 ± | 0.90 ± 0.03^{f} | 1.53 ± 0.06^{b} | 0.98 ± | 0.93 ± 0.06^{e} | 0.66 ± | 2.90 ± 0.03^{a} | 0.88 ± 0.05^{f} | 1.43 ± 0.02^{c} |
| tannins | 0.03^{d} | 0.04^{e} | 0.04^{g} | 0.05^{a} | | | 0.04^{d,e} | | 0.04^{g} | | | |
| (mg/g) | | | | | | | | | | | | |
| TIA* (U) | 0.75 ± | 0.83 ± | 0.81 ± | 0.85 ± | 0.77 ± 0.02^{a} | 0.43 ± 0.02^{e} | 0.53 ± 0.02^{d} | 0.54 ± 0.02^{c} | 0.63 ± | 0.64 ± 0.02^{b} | 0.55 ± 0.03^{c,d} | 0.19 ± 0.01^{f} |
| | 0.05^{a} | 0.03^{a} | 0.03^{a} | 0.03^{a} | | | | | 0.03^{b,c} | | | |
| Phytic acid | 1.56 ± | 2.23 ± | 2.53 ± | 2.36 ± | 2.64 ± 0.06^{a} | 1.23 ± 0.05^{e} | 0.60 ± 0.13^{g} | 1.59 ± 0.20^{c,d} | 1.07 ± | 1.40 ± 0.23^{d} | 1.85 ± 0.05^{c} | 0.70 ± 0.15^{g} |
| (g/100 g) | 0.07^{d} | 0.06^{b} | 0.09^{a} | 0.11^{b} | | | | | 0.18^{f} | | | |
| Phytasic | 3.10 ± | 2.84 ± | 1.61 ± | 2.05 ± | 2.05 ± 0.04^{h} | 1.66 ± 0.03^{l} | 3.71 ± 0.06^{a} | 3.54 ± 0.03^{b} | 2.60 ± | 2.65 ± 0.04^{e} | 2.55 ± 0.02^{f} | 2.47 ± 0.03^{g} |
| activity | 0.02^{c} | 0.03^{d} | 0.04^{l} | 0.03^{h} | | | | | 0.03^{e} | | | |
| (U)** | | | | | | | | | | | | |
| Total | 0.64 ± | 0.83 ± | 1.04 ± | 1.47 ± | 0.96 ± 0.02^{b} | 0.73 ± 0.01^{d} | 0.31 ± 0.02^{g} | 0.63 ± 0.02^{a} | 0.83 ± | 0.73 ± 0.02^{d} | 0.71 ± 0.03^{d} | 0.40 ± 0.01^{f} |
| saponins | 0.02^{e} | 0.02^{c} | 0.02^{b} | 0.05^{a} | | | | | 0.02^{c} | | | |
| (mg/g) | | | | | | | | | | | | |

^{a-f}Values in the same row with different superscript letters are significant y different (P <0.05). The data are the result of three independent experiments ± standard deviation (n = 3).
*Trypsin inhibitor activity, expressed as units of inhibition/mg of sample.
**The activity of a unit (U) is expressed as the amount of enzyme required to liberate 1 μmol/min of p-nitrophenyl under the test conditions.

### Example 2

Selection of Lactic Bacteria Included in the Invention, Use and Characterisation of the Fermentation Process

Materials and Methods

Selection of Lactic Bacteria

Lactobacillus plantarum DSM 33412 and Lactobacillus brevis DSM 33413 (which was deposited with the Deutsche Sammlung von Mikroorganismen and Zellkulturen GmbH (German Collection of Microorganisms and Cell Cultures) on the date of 22 Jan. 2020) were compared for pro-technological performance to nine microorganisms previously characterised and selected as starters. The latter, preserved in the Crop Collection of the Department of Soil Science of the University of Bari, are listed below: Lactobacillus plantarum T0A10 (Rizzello et al., 2016. Food Microbiology, 56:1-13), Lb. plantarum 18S9, Pediococcus acidilactici 10MMM0 and Leuconostoc mesenteroides 12MM1 (Nionelli et al., 2018. International Journal of Food Microbiology, 279:14-25), Lb. plantarum LB1 and Lactobacillus rossiae LB5 (Rizzello, et al., 2010. Food Chemistry, 119:1079-1089), e Lb. plantarum MRS1, MR10, and Lactobacillus brevis MRS4 (Rizzello et al., 2014. International Journal of Food Microbiology, 180:78-87). The strains were previously isolated from flour and spontaneously fermented doughs of quinoa, hemp, wheat germ and chickpea (Table 6).

TABLE 6
Lactic bacteria strains used as a reference in the
selection process and relevant source of isolation.

| Strain | Name | Source | References |
|---|---|---|---|
| Lactobacillus plantarum | T0A10 | Quinoa flour | Rizzello et |
| | | | al., 2016 |
| Lactobacillus plantarum | 18S9 (s5) | Spontaneously | |
| | | fermented hemp | |
| Pediococcus acidilactici | 10MM0 (s5) | Hemp flour | Nionelli et |
| | | | al., 2018 |
| Leuconostoc | 12MM1 (s1) | Spontaneously | |
| mesenteroides | | fermented hemp | |
| Lactobacillus plantarum | MRS1 | Spontaneously | Rizzello et |
| Lactobacillus plantarum | MRS10 | fermented | al., 2014 |
| Lactobacillus brevis | MRS4 | chickpea | |
| Lactobacillus plantarum | LB1 | Wheat germ | Rizzello. et |
| Lactobacillus rossiae | LB5 | | al., 2010 |

The strains covered by the present invention and the nine reference strains were used as starters for fermenting legume doughs produced with the two flour variants (s and sg) in order to compare the pro-technological performance thereof.

LAB strains were propagated in MRS (broth) at 30° C. for 24 hours. The cells were collected by centrifugation (10000 rpm, 10 min, 4° C.), washed twice in sterile potassium phosphate buffer (50 mM, pH 7.0), resuspended in water at a cell density of about 8.0 log 10 cfu/ml and used as a starter for the fermentation of doughs produced with legume flours (initial cell density of the dough, about 7.0 log 10 cfu/g), with the aim of monitoring the main pro-technological characteristics. The water:flour ratio of the doughs was selected on the basis of water absorption data obtained by analysis with the Brabender farinograph, and expressed as dough yield (DY, dough weight×100/flour weight) (Table 7). The fermentation was carried out in triplicate at 30° C. for 24 hours.

TABLE 7
Dough yield* of doughs based on unprocessed
(s) and processed (sg) legume flours.

Dough Yield*

| Flour | Symbol | s | sg |
|---|---|---|---|
| Red lentil | Lr | 168 | 192 |
| Yellow lentil | Ly | 176 | 192 |
| Chickpea | Cp | 176 | 192 |
| White bean | Bw | 176 | 192 |
| Black bean | Bb | 166 | 208 |
| Pea | Pe | 160 | 192 |

Wherein:

$${}^{*} Dough Yield = \frac{peso impasto}{peso sfarinato} {}^{*} 100$$

The growth and acidification kinetics were determined and modelled according to the Gompertz equation, modified by Zwietering et al., 1990 (Zwietering et al., 1990. Applied and Environmental Microbiology, 56:1875-1881): y=k+A exp{-exp[(p or Vmax e/A)(−t)+1]}, where y represents the growth expressed as log cfu/g/h or the acidification rate expressed as dpH/Δt (pH/h unit) at time t; k is the initial level of the dependent variable to be modelled (log cfu/g or pH unit); A is the change in cell density or pH (units) (between inoculum and stationary step); μmax or Vmax is the maximum growth rate expressed as dlog cfu/g/h or the maximum acidification rate expressed as dpH/dh, respectively; λ is the length of the latency step measured in hours. The experimental data were modelled as a non-linear regression using Statistica 12.0 software (Statsoftlnc., Tulsa, USA).

The pH was determined with a pH meter (model 507, Orison, Milan, Italy) with a food penetration probe.

Results

Doughs with a semi-solid consistency (compact, homogeneous and workable dough) based on legume flour and water were prepared by modulating the water additions according to the analyses previously carried out with the Brabender farinograph. Due to the different water absorption capacity, the DY ranged from 160 to 176 for s while it was higher in the sg flours, from 192 to 208 (Table 7). All doughs were inoculated individually with the two microorganisms included in the present invention and the nine comparison strains. The growth and acidification kinetics were monitored during 24 hours of incubation at 30° C. During the fermentation process, a growth of about 2 log 10 cycles was observed in all matrices (final cell density equal to 9.1±0.5-9.6±0.4 log 10 cfu/g dough). The kinetic parameters of the acidification process obtained for both s and sg strains were evaluated (Table 8).

L. plantarum DSM 33412 and L. brevis DSM 33413 showed the most intense acidifying activity (median dpH values equal to 2.82±0.1 and 2.79±0.2, respectively) (Table 8). Among the other nine strains used in the evaluation, the highest median value was found for L. plantarum MRS1 (2.41±0.1), while all others were below 2. The highest dpH values were found for both L. plantarum DSM 33412 and L. brevis DSM 33413 for the doughs based on red lentil and pea, while the lowest values were observed for chickpea doughs.

The acidification latency step (A) varied over a wide range for all micro-organisms considered (0.20-8.97 hours) with the exception of L. plantarum DSM 33412 and L. brevis DSM 33413, for which the entire distribution of values never exceeded 1.5 hours (Table 8). In particular, the median values were 0.39±0.02 and 0.50±0.03 for L. plantarum DSM 33412 and L. brevis DSM 33413 respectively.

L. plantarum DSM 33412 and L. brevis DSM 33413 also showed the highest acidification rate (median Vmax values equal to 0.54 and 0.51, respectively), with great uniformity of performance on all matrices considered (Table 8). Unlike all other strains considered in the evaluation, the Vmax values of the two strains were in fact never lower than 0.50.

The acidification kinetics are considered to be an index of the protechnological characteristics and the adaptation of the strain to the matrix to be fermented.

TABLE 8
Parameters of the acidification kinetics (dpH; λ(h), Vmax
(dpH/h)) of the strains of lactic bacteria used to ferment
doughs from legume flours (red lentil, yellow lentil, white
bean, black bean, chickpea and pea, as such and after the technological
process) at 30° C. for 24 hours. In particular, the minimum
(Min), maximum (Max) and median values of the entire distribution
are reported for each of the strains. All individual data refer
to three independent experiments (n = 3).

| strain | min | Max | Median |
|---|---|---|---|
| | dpH | | |
| DSM 33412 | 2.54 | 3.89 | 2.82 |
| DSM 33413 | 2.41 | 3.61 | 2.79 |
| MRS1 | 1.94 | 3.47 | 2.42 |
| MRS4 | 0.56 | 2.76 | 1.53 |
| MRS10 | 1.57 | 2.18 | 1.66 |
| LB1 | 1.68 | 2.35 | 1.78 |
| LB5 | 1.73 | 2.39 | 2.08 |
| 18S9 | 1.90 | 2.16 | 2.03 |
| 12MM1 | 1.74 | 2.28 | 1.83 |
| 10MM0 | 1.59 | 2.33 | 1.87 |
| T0A10 | 1.79 | 2.12 | 1.91 |
| | λ(h) | | |
| DSM 33412 | 0.21 | 1.51 | 0.40 |
| DSM 33413 | 0.32 | 1.32 | 0.50 |
| MRS1 | 0.36 | 6.95 | 1.08 |
| MRS4 | 0.33 | 8.54 | 8.12 |
| MRS10 | 0.20 | 2.19 | 1.19 |
| LB1 | 0.28 | 8.63 | 7.83 |
| LB5 | 0.21 | 8.12 | 0.28 |
| 18S9 | 0.20 | 4.27 | 0.30 |
| 12MM1 | 1.32 | 8.97 | 7.39 |
| 10MM0 | 0.21 | 8.77 | 2.40 |
| T0A10 | 0.44 | 3.78 | 1.39 |
| | Vmax (dpH/h) | | |
| DSM 33412 | 0.50 | 0.58 | 0.54 |
| DSM 33413 | 0.50 | 0.55 | 0.52 |
| MRS1 | 0.44 | 0.52 | 0.46 |
| MRS4 | 0.11 | 0.24 | 0.22 |
| MRS10 | 0.42 | 0.52 | 0.44 |
| LB1 | 0.43 | 0.52 | 0.48 |
| LB5 | 0.10 | 0.32 | 0.11 |
| 18S9 | 0.33 | 0.55 | 0.48 |
| 12MM1 | 0.12 | 0.25 | 0.23 |
| 10MM0 | 0.34 | 0.51 | 0.46 |
| T0A10 | 0.35 | 0.52 | 0.49 |

On the basis of the results obtained, the strains L. plantarum DSM 33412 and L. brevis DSM 33413 were considered to be the best performing under the application conditions of interest of the present invention and better than microorganisms previously selected as starters for the fermentation of legume matrices, included as reference in the present comparative evaluation.

The characterisation analyses performed on the matrices fermented with the two selected strains are shown in the following examples.

### Example 3

Nutritional Characterisation of Pre-Gelatinised Flours Fermented with Selected Starters (in Comparison with Unprocessed Matrices)

Materials and Methods

Conditions of the Fermentation Process

On the basis of the parameters of the growth and acidification kinetics reported in Example 2, it is shown that Lb. plantarum DSM 33412 and Lb. brevis DSM 33413 show a higher pro-technological performance than the reference strain cluster.

The two strains were then used as a mixed starter (1:1 ratio) for the fermentation of s and sg. The cell suspensions and doughs were prepared as described in Example 2. The fermentation process was carried out in triplicate, at 30° C. for 24 hours.

Nutritional Characterisation of Fermented Pre-Gelatinised Flours

The flours unprocessed and fermented by means of fermentation process (sF), and fermented pre-gelatinised flours (sgF), by means of the technological process and fermentation process of the invention, were characterised and compared by microbiological analysis and for organic acid content and fermentation quotient (QF), total polyphenols, antioxidant activity (determined on DPPH radical), total free amino acids (TFAA), in vitro protein digestibility (IVPD), resistant starch (RS), starch hydrolysis (HI) and dietary fibre, as described in Example 1. In addition, they were also characterised for content in anti-nutritional factors, such as raffinose, condensed tannins, phytic acid, saponins and trypsin and phytasic inhibition activity, as described in Example 1.

The data were also compared with the corresponding unfermented s and sg flours of Example 1 (Tables 3-5).

Dough Fermentation

The doughs inoculated with the selected starters were incubated for 24 hours at 30° C. A growth of about 2 log 10 was observed for the starter lactic bacteria in all samples (cell density equal to 9.75±0.05-9.85±0.04 log 10 cfu/g). As a consequence of lactic fermentation, the pH decreased from 4.15±0.22 to 4.54±0.13 for sF, and slightly lower values were observed for sgF (from 4.11±0.21 to 4.36±0.24) (FIG. 2A). No significant differences were found between the different legume flours (P>0.05). The lactic acid, which as expected was not found in the unfermented flours, reached concentrations of up to about 120 mmol/kg in the fermented doughs. The median values for sF and sgF fermented flours were 103.8±4.0 and 106.1±3.6 mmol/kg, respectively (FIG. 2B). The highest concentrations were found in chickpea doughs. Although only slight differences were found in the final lactic acid concentration of sF and sgF, the acetic acid synthesis significantly (P<0.05) differed for all legume flours considered, between sF and sgF (FIG. 2C). In sF the concentration of acetic acid ranged from 14.3±0.2 to 28.3±0.1 while it ranged from 21.7±1.1 to 44.4±3.3 mmol/kg in sgF. The greatest differences were observed for the doughs obtained from red and yellow lentil, and black bean sgF, characterised by acetic acid concentrations 2 to 4 times higher than those found in the corresponding sF. The FQ value ranged from 4.10±0.24 to 6.67±0.41 for sF, while values between 2.16±0.05 and 4.63±0.21 were found for sgF.

Total Phenols and Antioxidant Activity

An increase in total phenols in both sF and sgF was observed during fermentation.

The highest increase was observed in the red and yellow lentil doughs sF, while the chickpea dough sF contained the highest amount of total phenols (2.74±0.02 mmol/kg). In addition, the concentration was 2-3 times higher than in the corresponding unfermented s and sg flours.

The antioxidant activity was highest in the doughs sF (Table 9).

In detail, the highest activity was observed for black bean, both sF and sgF (96.0±1.4% and 84.3±1.0% respectively).

Also in this case, the antioxidant activity was higher than that of the corresponding s and sg flours.

Proteolysis and IVPD

Fermentation caused a significant increase in the concentration of TFAA in both sF and sgF.

In fact, when compared to the corresponding s and sg flours, an increase from 22 to 65% and up to 76% was observed in sF and sgF, respectively (Table 9).

With the exception of the pea flour dough, all doughs sgF were characterised by a TFAA concentration above about 2.3 g/kg.

After fermentation, a further increase in IVPD was found in all samples, with the highest values being found in sgF. In fact, digestibility was >ca. 88% for all sF, while sgF were characterised by IVPD close to 100% (Table 9).

Resistant Starch, Starch Hydrolysis and Dietary Fibre

After fermentation, significant (P<0.05) increases were found for resistant starch in the sF samples when compared to the corresponding s flours (final values 2-3 times higher than in the unfermented samples).

Even higher values were observed in the pre-gelatinised and fermented doughs sgF (8.45±0.06-18.97±0.51%) (Table 9).

Both the technological and fermentation processes caused a decrease in HI.

The combination of the processings, in particular, led to a further significant reduction in HI values in sgF.

Overall, HI values ranged from 32% (chickpea sgF) to 50% (black and white bean sgF) (Table 9).

Overall, fermentation caused an increase in SDF, IDF and TDF (Table 10) in all samples, although the greatest increases were found in the pre-gelatinised samples.

The soluble fibre SDF (Table 10) in sF and sgF ranged from 6.1±0.4 to 11.1±0.4% and from 6.5±0.4 to 12.1±0.3% respectively, and the values were significantly (P<0.05) higher than the corresponding s and sg (Table 4).

In detail, the largest increases (final concentration up to 2 times higher than the initial concentration) were found for yellow lentil doughs, while more moderate increases (38-62%) were observed in all the other cases. The same trend was observed for IDF (Table 10). Compared to unfermented s and sg (Table 4), the highest IDF concentrations were found for white (28.3±0.4%) and black bean (25.4±0.5%). The total fibre TDF concentrations in sF ranged from 24.1±0.4 to 32.2±0.4%, of which white and black bean flours had the highest content (32.2±0.4 and 32.1±0.4% respectively). Higher average values, in the range of 25.0±0.5-35.2±0.5%, were found in sgF, among which the highest content was observed in white and black bean flours (Table 10).

Antinutritional Factors

Raffinose

Raffinose concentrations decreased significantly with the fermentative processes. When the fermentation with starters was applied to unprocessed flours (s), the decreases were 70-82%.

When the technological and fermentation process (sgF) were applied sequentially, the raffinose concentration in the doughs was significantly lower than in the corresponding doughs sF, with decreases of up to 89% in the concentration compared to unfermented gelatinised flours (Table 11).

Condensed Tannins

After fermentation, a significant (P<0.05) decrease in the concentration of condensed tannins was observed in both sF and sgF, depending on the flours considered.

In fact, it decreased from 70 to 94% and from 75 to 95% respectively in sF and sgF, when compared to the corresponding s and sg (Table 11).

However, considering that the flours that have undergone the technological process (sg) have a significantly lower starting quantity of s, a final concentration of condensed tannins in sgF of less than 0.25 mg/g was observed in almost all cases.

Trypsin Inhibitor Activity

The fermentation with the starters L. plantarum DSM 33412 and L. brevis DSM 33413 resulted in a decrease in trypsin inhibitor activity between 35 and 75% in sF (compared to the same unfermented s flours)(Table 11).

The activity reduction is equal to 100% when the fermentation is applied to gelatinised flours (Table 11), i.e., with the combination of technological and fermentation processings (sgF vs. s)

Phytic Acid and Phytasic Activity

With the fermentation process (sF and sgF), the phytic acid is completely degraded in all samples, with a 100% decrease in each case considered (Table 11).

The phytase activity increased significantly (P>0.05) as a result of both technological and fermentation processing, reaching significantly (P<0.05) higher values when the processes were combined (sgF).

Total Saponins

Fermentation with the strains L. plantarum DSM 33412 and L. brevis DSM 33413 resulted in a decrease of saponins in the non-technologically processed flours in the range of 72 to 86% (sg vs. s) (Tables 5 and 11).

The combined processing of the two processes resulted in a 100% decrease in the concentration of total saponins in SgF flours (Table 11).

### Concluding Remarks

The fermentation with the strains L. plantarum DSM 33412 and L. brevis DSM 33413 proves to be an effective processing for improving the nutritional profile and reducing anti-nutritional factors of legume flours.

The scientific and technical literature does not so far report results of the same magnitude as those obtainable with the fermentation protocol described in the present invention.

When fermentation is applied to non-technologically processed flours, significant increases (P<0.05) were found in: total polyphenol concentration and antioxidant activity, free amino acid concentration, protein digestibility and resistant starch concentration.

The increase in polyphenolic compounds is linked to the metabolic characteristics of the selected lactic bacteria included in this patent: through their enzymatic endowment they are able to release simple molecules from more complex or glycosylated molecules. Their increase in the matrix leads to an increase in the antioxidant activity, the polyphenols being molecules responsible for scavenging free radicals and oxidant or pro-oxidant species. This characteristic is currently being sought in the formulation of new foodstuffs or for the improvement of conventional ones, given the concrete scientific evidence showing that the supply of molecules with antioxidant activity can prevent degenerative phenomena and tissue ageing (anti-radical and anti-ageing action).

The increase in free amino acids, linked to the proteolytic activity of the lactic bacteria included in this patent (this activity has been considered as a comparative selection criterion with respect to other known starters) is, as already mentioned above, of great nutritional importance (they are readily assimilable, unlike proteins which require the intervention of digestive enzymes) and sensory importance, with a direct effect on the taste profile (sapidity) and on the complexity of the olfactory profile of food products containing them. The intensive proteolytic activity operated by the selected lactic bacteria during the fermentation process also results in an increase in protein digestibility: these micro-organisms are able, through their enzymes (proteases and peptidases) to break down proteins into polypeptides, peptides and free amino acids, which in fact corresponds to a pre-digestion of the matrix proteins. It has now been widely demonstrated (through in vivo clinical trials) that this facilitates and speeds up the digestive process, which occurs more quickly and with a reduced perception of fatigue.

Finally, the increase in resistant starch, made possible by the biological acidification that occurs during the fermentation process, leads to a reduction in the glycaemic index, which is of great importance in balancing the diet of the modern consumer (need for reduced supply of simple carbohydrates).

In addition, the sequential combination of the technological processing with fermentation under controlled conditions enables a further reduction in anti-nutritional compounds, which is already partially achievable with the technological process alone. There are significant further decreases in raffinose and condensed tannin levels, with complete degradation of trypsin inhibitors, phytic acid and saponins.

It should be emphasised that the abundance of anti-nutritional compounds in gluten-free raw materials, such as legumes and pseudocereals, is currently the major cause of limitation to their large-scale use in the food sector. The combined solution (technological process and fermentation) described in this patent is the only one of the various processings proposed by the technical and scientific community (soaking, germination, fractioning, roasting) capable of guaranteeing a reduction of this magnitude in all anti-nutritional compounds at the same time. While the reduction in raffinose reduces the bloating and flatulence phenomena associated with the consumption of legumes, the reduction in condensed tannins, phytic acid, trypsin inhibitors and saponins allows to increase the bioavailability of proteins and mineral salts, enhancing the potential of native raw material.

Compared to pre-gelatinised flours at the end of the technological process, fermentation results in an increase in total polyphenols (by 2-3 times) and in antioxidant activity, in the concentration of free amino acids (up to 76%), in protein digestibility (with final values of 100%), and in resistant starch (up to 2-3 times) compared to unfermented flours. In addition, the starch hydrolysis index, a parameter related to the in vivo glycaemic index, was below 55% in all cases.

TABLE 9
Concentration of total polyphenols, antioxidant activity (determined on DPPH radical), total free amino acids (TFAA) in vitro
protein digestibility (IVPD), resistant starch and starch hydrolysis (HI) in fermented flours using
Lactobacillus plantarum DSM 33412 and Lactobacillus
brevis Dsm 33413 as starters, at 30° C. for 24 h. The fermentation was applied to both unprocessed flours with a technological
process non-gelatinised sf) and flours subjected to technological processing (fermented gelatinised flours - sgF)

| | sF | | | | | | sgF | | | | | |
|---|---|---|---|---|---|---|---|---|---|---|---|---|
| | Red | Yellow | White | Black | Chick- | | Red | Yellow | White | Black | | |
| | Lentil | Lentil | Bean | Bean | pea | Pea | Lentil | Lentil | Bean | Bean | Chickpea | Pea |
| Total | 1.72 ± | 1.93 ± | 1.55 ± | 1.83 ± | 2.74 ± | 2.15 ± | 1.24 ± | 1.33 ± 0.04 [symbol] | 1.16 ± | 1.31 ± | 2.02 ± | 1.55 ± |
| phenols | 0.02^{d} | 0.02^{c} | 0.01^{e} | 0.05^{c} | 0.02^{a} | 0.04^{b} | 0.01^{g} | | 0.03^{g} | 0.01^{f} | 0.10^{b} | 0.02^{a} |
| (mmol/kg) | | | | | | | | | | | | |
| DPPH (%) | 58.9 ± | 69.1 ± | 67.3 ± | 96.0 ± 1.4^{a} | 56.8 ± | 59.0 ± | 57.5 ± | 63.2 ± 1.1^{d} | 50.2 ± | 84.3 ± | 47.4 ± | 53.7 ± |
| | 1.4^{a} | 1.2^{c} | 1.7^{c} | | 1.3^{e} | 1.0^{e} | 1.2^{a} | | 1.5^{f} | 1.0^{b} | 1.3^{g} | 1.4^{f} |
| TFFA | 2600 ± | 2594 ± | 2109 ± | 1981 ± 84 [symbol] | 2401 ± | 1595 ± | 2797 ± | 2993 ± 130^{a} | 2319 ± | 2543 ± | 2815 ± | 1935 ± |
| (mg/kg) | 115^{c} | 103^{c} | 95^{a,e} | | 113 [symbol] | 78^{f} | 121^{b} | | 1.00^{d} | 127^{c} | 79^{b} | 81^{e} |
| IVPD (%) | 79 ± 3^{c} | 91 ± 1^{a,b} | 88 ± 2^{c} | 88 ± 3^{c} | 97 ± 2^{a,b} | 89 ± 2^{c} | 99 ± 1^{a} | 99 ± 1^{a} | 99 ± 1^{a} | 99 ± 1^{a} | 99 ± 1^{a} | 99 ± 1^{a} |
| Resistant | 7.01 ± | 7.34 ± | 15.5 ± | 16.76 ± | 7.19 ± | 6.74 ± | 8.66 ± | 8.74 ± 0.12^{d} | 17.96 ± | 18.97 ± | 8.451 ± | 9.58 ± |
| starch (%) | 0.16^{e} | 0.05^{e} | 0.57^{b} | 0.71^{b} | 0.06^{e} | 0.11^{f} | 0.14^{d} | | 0.77^{a} | 0.51^{a} | 0.06^{d} | 0.42^{c} |
| HI (%) | 45 ± 1^{c} | 47 ± 2^{c} | 53 ± 1^{a} | 53 ± 1^{a} | 35 ± 1^{a} | 55 ± 2^{a} | 42 ± 2^{g} | 43 ± 2^{d} | 50 ± 2^{c} | 50 ± 1^{b} | 32 ± 2^{e} | 47 ± 2^{c} |

^{a-g}Values in the same row with different superscript letters are significantly different (P <0.05). The data are the result of three independent experiments ± standard deviation (n = 3). [symbol]
indicates data missing or illegible when filed

TABLE 10
Soluble dietary fibres (SDF), insoluble dietary fibres (IDF) and total dietary fibres (TDF) in flours fermented using Lactobacillus plantarum DSM
33412 and Lactobacillus brevis DSM 33413 as starters, at 30° C. for 24 h. The fermentation was applied to both unprocessed flours with technological
process (fermented - non-gelatinised sf) and flours subjected to technological processing (fermented gelatinised flours - sgF) Within the
analytical category (SDF, IDF and TDF), values labelled with different letters differed significantly (P <0.05).

| | SDF | | IDF | | TDF | |
|---|---|---|---|---|---|---|
| | sF | sgF | sF | sgF | sF | sgF |
| Red lentil | 8.1 ± 0.2^{b} | 8.3 ± 0.4 [symbol] | 18.2 ± 0.4 [symbol] | 20.4 ± 0.8^{c} | 26.1 ± 0.4^{c} | 28.3 ± 0.4 [symbol] |
| Yellow lentil | 6.1 ± 0.4^{c} | 6.5 ± 0.4^{c} | 18.1 ± 0.3^{d} | 18.5 ± 0.6^{d} | 24.1 ± 0.4^{d} | 25.4 ± 0.5^{e} |
| White bean | 6.6 ± 0.3^{c} | 6.9 ± 0.5^{c} | 25.4 ± 0.4^{a} | 28.3 ± 0.4^{a} | 32.2 ± 0.4^{a} | 35.2 ± 0.5^{a} |
| Black bean | 8.3 ± 0.4^{b} | 8.7 ± 0.4^{b} | 23.7 ± 0.6^{b} | 25.4 ± 0.5^{b} | 32.1 ± 0.4^{ab} | 34.1 ± 0.3^{b} |
| Chickpea | 11.1 ± 0.4^{a} | 12.1 ± 0.3^{a} | 20.4 ± 0.4^{c} | 21.4 ± 0.4^{c} | 31.4 ± 0.6^{b} | 33.5 ± 0.7^{b} |
| Pea | 11.1 ± 0.2^{a} | 11.7 ± 0.5^{a} | 15.4 ± 0.2^{e} | 18.7 ± 0.4^{d} | 26.4 ± 0.4^{c} | 30.4 ± 0.6^{c} |

[symbol]

indicates data missing or illegible when filed

TABLE 11
Raffinose, condensed tannins, phytic acid, saponins and trypsin and phytasic inhibition activity in fermented flours using
Lactobacillus plantarum DSM 33412 and Lactobacillus brevis DSM 33413 as starters, at 30° C.
for 24 h. The fermentation was applied to both unprocessed flours with a technological process
(fermented - non-gelatinised sf) and flours subjected to technological processing (fermented gelatinised flours - sgF)

| | sF | | | | | | sgF | | | | | |
|---|---|---|---|---|---|---|---|---|---|---|---|---|
| | | Yellow | White | Black | | | Red | Yellow | White | Black | Chick- | |
| | Red Lentil | Lentil | Bean | Bean | Chickpea | Pea | Lentil | Lentil | Bean | Bean | pea | Pea |
| Raffinose | 0.53 ± 0. [symbol] 01^{b} | 0.65 ± | 0.10 ± | 0.35 ± 0.02 [symbol] | 0.41 ± 0.02^{d} | 0.65 ± | 0.52 ± | 0.61 ± | 0.14 ± | 0.13 ± | 0.59 ± | 0.48 ± |
| (g/kg) | | 0.03^{a} | 0.01^{f} | | | 40.03 | 0.02 [symbol] | 0.01^{a} | 0.02 [symbol] | 0.04^{f} | 0.07 [symbol] | 0.02 [symbol] |
| Condensed | 0.32 ± 0.07^{b} | 0.29 ± | 0.03 ± | 1.93 ± 0.01^{a} | 0.06 ± 0.02 [symbol] | 0.16 ± | 0.25 ± | 0.20 ± | 0.02 ± | 1.81 ± | 0.01 ± | 0.10 ± |
| tannins | | 0.05^{b} | 0.02^{ef} | | | 0.2^{d} | 0.03 [symbol] | 0.02^{cd} | 0.02 [symbol] | 0.07^{a} | 0.01^{f} | 0.03 [symbol] |
| (mg/g) | | | | | | | | | | | | |
| TIA* (U) | nd | nd | nd | nd | nd | nd | nd | nd | nd | nd | nd | nd |
| Phytic acid | nd | nd | nd | nd | nd | nd | nd | nd | nd | nd | nd | nd |
| (g/100 g) | | | | | | | | | | | | |
| Phytasic | 4.34 ± 0.07^{d} | 6.02 ± | 3.12 ± | 2.91 ± 0.07 [symbol] | 3.51 ± 0.08 [symbol] | 5.12 ± | 5.80 ± | 6.00 ± | 4.01 ± | 3.75 ± | 5.82 ± | 6.01 ± |
| activity (U)** | | 0.06^{a} | 0.05^{h} | | | 0.03^{c} | 0.09^{b} | 0.06^{a} | 0.03 [symbol] | 0.07 [symbol] | 0.08^{b} | 0.08^{a} |
| Total | 0.10 ± 0.01^{a} | 0.21 ± | 0.30 ± | 0.41 ± 0.03 [symbol] | 0.33 ± 0.02 [symbol] | 0.11 ± | nd | nd | nd | nd | nd | nd |
| saponins | | 0.02^{d} | 0.05^{a} | | | 0.02 [symbol] | | | | | | |
| (mg/g) | | | | | | | | | | | | |

^{aH}Values in the same row with different superscript letters are significantly different (P <0.05). The data are the result of three independent experiments ± standard deviation (n = 3).
*Trypsin inhibitor activity, expressed as units of inhibition/mg of sample.
**The activity of a unit (U) is expressed as the amount of enzyme required to liberate 1 μmol/min of p-nitrophenyl under the trial conditions nd: not detectable. [symbol]
indicates data missing or illegible when filed

Materials and Methods

Obtaining dehydrated fermented flowing material.

S- and sg-type flours were fermented with the selected strains Lactobacillus plantarum DSM 33412 and Lactobacillus brevis DSM 33413. The cell suspensions and doughs were prepared as described in Example 2. The fermentation process was carried out in triplicate, at 30° C. for 24 h, as described in example 3.

All the fermented doughs (sf and sgf) were i) refrigerated at a temperature between 4 and 12° C. ii) frozen at −20° C.; iii) dehydrated by lyophilization, after freezing at −50° C.; iV) dried in a ventilated oven at 65° C. for 1.5 h. The cell density of the lactic bacteria was analysed 6 and 12 months after stabilisation by freezing and dehydration, by plate counting on MRS supplemented with cycloheximide (0.1 g/l), as described in example 1. The refrigerated samples, on the other hand, were analysed after 7 days of storage, which is compatible with the correct management of the hygiene and health standards of food and food ingredients. The refrigerated samples showed a cell density of lactic bacteria in the range of 2-6×108 cfu/g at 7 days.

The results of microbiological analysis of samples stabilised by freezing and dehydration are shown in Table 12.

TABLE 12
Density of lactic bacteria (cfu/g) in flours fermented using Lactobacillus plantarum DSM 33412 and Lactobacillus
brevis DSM 33413 as starters, and then stabilised by freezin g or dehydration (lyophilization, drying at 65° C). Fermentation
and subsequent stabilisation processings were applied to both the unprocessed flours with technological process
(fermented - non-gelatinissed sf) and the flours subjected to technological processing (fermented gelatinised flours - sgF)

| | freezing | | | | lyophilization | | | | drying\| | | | |
|---|---|---|---|---|---|---|---|---|---|---|---|---|
| | sF | | sgF | | sF | | sgF | | sF | | sgF\| | |
| | 6 | 12 | 6 | 12 | 6 | 12 | 6 | 12 | 6 | 12 | 6 | 12 |
| | months | months | months | months | months | months | months | months | months | months | months | months |
| Red | 6 × | 4 × | 5 × 10 [symbol] | 4 × 10 [symbol] | 5 × 10 [symbol] | 3 × 10 [symbol] | 6 × 10 [symbol] | 3 × 10 [symbol] | 4 × 10 [symbol] | 2 × 10 [symbol] | 4 × 10 [symbol] | 2 × 10 [symbol] |
| lentil | 10 [symbol] | 10 [symbol] | | | | | | | | | | |
| Yellow | 5 × | 5 × | 6 × 10 [symbol] | 5 × 10 [symbol] | 5 × 10 [symbol] | 5 × 10 [symbol] | 5 × 10 [symbol] | 5 × 10 [symbol] | 5 × 10 [symbol] | 5 × 10 [symbol] | 4 × 10 [symbol] | 5 × 10 [symbol] |
| lentil | 10 [symbol] | 10 [symbol] | | | | | | | | | | |
| White | 7 × [symbol] | 6 × | 6 × 10 [symbol] | 5 × 10 [symbol] | 6 × 10 [symbol] | 3 × 10 [symbol] | 7 × 10 [symbol] | 3 × 10 [symbol] | 5 × 10 [symbol] | 3 × 10 [symbol] | 5 × 10 [symbol] | 3 × 10 [symbol] |
| bean | 10 | 10 [symbol] | | | | | | | | | | |
| Black | 5 × | 3 × | 5 × 10 [symbol] | 5 × 10 [symbol] | 5 × 10 [symbol] | 5 × 10 [symbol] | 5 × 10 [symbol] | 5 × 10 [symbol] | 5 × 10 [symbol] | 5 × 10 [symbol] | 4 × 10 [symbol] | 5 × 10 [symbol] |
| bean | 10 [symbol] | 10 [symbol] | | | | | | | | | | |
| Chick- | 6 × | 6 × | 5 × 10 [symbol] | 4 × 10 [symbol] | 5 × 10 [symbol] | 2 × 10 [symbol] | 6 × 10 [symbol] | 3 × 10 [symbol] | 5 × 10 [symbol] | 2 × 10 [symbol] | 5 × 10 [symbol] | 3 × 10 [symbol] |
| pea | 10 [symbol] | 10 [symbol] | | | | | | | | | | |
| Pea | 5 × | 4 × | 6 × 10 [symbol] | 4 × 10 [symbol] | 6 × 10 [symbol] | 2 × 10 [symbol] | 5 × 10 [symbol] | 3 × 10 [symbol] | 4 × 10 [symbol] | 2 × 10 [symbol] | 4 × 10 [symbol] | 2 × 10 [symbol] |
| | 10 [symbol] | 10 [symbol] | | | | | | | | | | |

[symbol]

indicates data missing or illegible when filed

### Concluding Remarks

Lactobacillus plantarum DSM 33412 and Lactobacillus brevis DSM 33413 show high survival to physical stabilisation processes of the flowing material. In particular, there is no decrease in viability in the frozen product over 12 months of storage (>2×109 cfu/g), and cell densities greater than 2×108 cfu/g in the case of products dehydrated by lyophilization or drying at low temperatures.

These conditions confirm the possibility of using stabilised fermented material as a biological fermentation and acidification agent in the preparation of oven-baked leavened products and other fermented foodstuffs.

1. Bacterial strain of Lactobacillus plantarum (F.1) which was deposited with the culture collection Deutsche Sammlung von Mikroorganismen und Zellkulturen GmbH (German Collection of Microorganisms and Cell Cultures) on Jan. 22, 2020 and which is identified by the deposit number DSM 33412.

2. Bacterial strain of Lactobacillus brevis (F.4) which was deposited with the culture collection Deutsche Sammlung von Mikroorganismen und Zellkulturen GmbH (German Collection of Microorganisms and Cell Cultures) on Jan. 22, 2020 and which is identified by the deposit number DSM 33413.

3. Method for fermenting flowing material without gluten comprising:
a) a step of mixing said flowing material with drinking water, in which the material is present in the admixture at a weight/volume percentage in the range from 30 to 60%;
b) a subsequent step of inoculating an admixture of lactic bacteria comprising at least one strain selected from the strain of Lactobacillus plantarum according to claim 1 or a strain Lactobacillus brevis (F.4) which was deposited with the culture collection Deutsche Sammlung von Mikroorganismen and Zellkulturen GmbH (German Collection of Microorganisms and Cell Cultures) on the date of 22 Jan. 2020 and which is identified by the deposit number DSM 33413;
c) a subsequent step of fermentation at a temperature between 20 and 35° C. for a time between 8 and 24 hours until reaching a pH between 4.0 and 5.0 and a final cell density of the microorganisms in the range from 1 to 7×10⁹ cfu/ml;
d) a subsequent optional step of refrigeration, at a temperature between 4 and 12° C., or freezing, at temperatures less than or equal to −20° C.;
e) an optional step of dehydration, following the step c) or the step d), which may optionally be followed by grinding in order to obtain a flour with a fine granulometry f) an optional packaging step which may be after each of the steps c), d) or e), wherein the material obtained is packed in suitable packaging for food products.

4. The method according to claim 3, wherein said admixture of lactic bacteria comprises said strain of Lactobacillus plantarum and said strain of Lactobacillus brevis.

5. Method according to claim 3, wherein said admixture of lactic bacteria comprises said strain of Lactobacillus plantarum and said strain of Lactobacillus brevis in a ratio of between 1:1 and 10:1.

6. The method according to claim 3, wherein said admixture of lactic bacteria further comprises at least one bacteria belonging to at least one species selected from the group consisting of: Lactobacillus plantarum, Lactobacillus brevis, Lactobacillus rossiae, Lactobacillus sanfranciscensis, Pediococcus pentosaceus and Leuconostoc spp.

7. The method according to claim 3, wherein the inoculation is carried out in step b) using living and viable cells at the cell density between 1 and 5×10⁷ cfu/ml of matrix to be fermented.

8. The method according to claim 3, wherein in step b) for inoculation the matrix is brought to a temperature between 20 and 35° C.

9. The method according to claim 3, wherein the step of dehydration e) may be carried out by lyophilization or at temperatures less than 70° C.

10. The method according to claim 3, wherein the step of dehydration e) may be carried out by lyophilization or at temperatures less than 65° C.

11. The method according to claim 3, wherein the flowing material without gluten used in step a) is selected from the group consisting of:
bean, Phaseolus vulgaris L; pea, Pisum sativum L;
fava bean, Vicia faba L; lupin, Lupinus albus chickpea, Cicer arietinum L.; pigeon pea, Cajanus indicus; groundnuts, Arachis hypogaea L.; soya, Glycine max; lenticchia, Lens culinaris; chickling vetch, Lathyrus sativus; carob tree, Ceratonia siliqua, preferably, Cicer arietinum L., Lens culinaris;
amaranth, Amaranthus spp.; quinoa, Chenopodium quinoa;
buckwheat, Fagopyrum esculentum;
rice, maize, millet, teff, sorghum,
and mixtures thereof.

12. Method for processing granular food material without gluten comprising:
providing a desired quantity of granular food material without gluten;
a step of humidification of said granular material, wherein said granular material is humidified in order to obtain a granular material having a humidity (UG) between 10 and 20%;
a step of heating in order to heat the granular material up to a heating temperature (TH) between about 60 and about 100° C. by means of saturated vapor;
a feeding step for transferring said granular material from said heating step to a lamination step;
a lamination step, in which said granular material is laminated at a lamination pressure (PL) between 50 and 150 bar in order to obtain flowing material in the form of flakes;
a drying step, in which the flake-like flowing material is dried by means of hot air at a drying temperature TE) between 140 and 160° C. in order to obtain flowing material at a humidity (UE) between 5 and 20%;
a cooling step in which the flake-like flowing material is cooled to a cooling temperature (TC) between 5 and 15° C., wherein said cooling step is adjusted so that the flakes are cooled within a cooling time (tC) less than 60 sec.; and
carrying out the method according to claim 3.

13. The material obtained by the method according to claim 3, in the form of flour or flakes and comprising at least one bacterial strain selected from said bacterial strain of Lactobacillus plantarum or said strain of Lactobacillus brevis in viable form.

14. The material according to claim 13, wherein said at least one bacterial strain has a cell density>10⁹ cfu/g at the end of step c) or d) or a cell density>10⁸ cfu/g at the end of step e).

15. The material obtained by the method according to claim 3, in the form of flour or flakes and comprising said bacterial strain of Lactobacillus plantarum or said strain of Lactobacillus brevis in viable form and having a cell density>109 cfu/g at the end of step c) or d) or a cell density>108 cfu/g at the end of step e).

16. The material according to claim 13, and having at least one of the following characteristics:
protein digestibility>80%;
starch hydrolysis index<64%;
total free amino acid concentration>1.4 g/kg;
concentration of resistant starch>2.5%;
raffinose concentration<2 g/kg;
phytic acid concentration<1.85 g/100 g; or
concentration of condensed tannins<2.9 mg/g.

17. The material obtained by the method according to claim 12, in the form of flour or flakes and comprising at least one bacterial strain selected from said bacterial strain of Lactobacillus plantarum or said strain of Lactobacillus brevis in viable form, having at least one of the following characteristics:
protein digestibility>99%
starch hydrolysis index<50%
total free amino acid concentration>1.9 g/kg
concentration of resistant starch>8%
raffinose concentration<0.6 g/kg
absence of phytic acid (0%), or
concentration of condensed tannins<1.8 mg/g.

18. Use of the material according to claim 13 as an acidifying agent in the preparation of oven-baked products.

19. Use of the strain according to claim 1 or a strain of Lactobacillus brevis (F.4) which was deposited with the culture collection Deutsche Sammlung von Mikroorganismen and Zellkulturen GmbH (German Collection of Microorganisms and Cell Cultures) on the date of 22 Jan. 2020 and which is identified by the deposit number DSM 33413: or mixtures thereof for fermenting flowing material without gluten.
